(19) United States
(12) Patent Application Publication
(10) Pub. No.: US 2009/0258411 A1
(43) Pub. Date: Oct. 15, 2009

(54) CENTRIFUGAL DEVICE AND METHOD FOR OVA DETECTION

(75) Inventors: Henry Petithory, Southborough, MA (US); James Kelland, E. Walpole, MA (US); Kevin Sullivan, Chestnut Hill, MA (US); Linda Martin, Norfolk, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: STATSPIN, INC. D/B/A IRIS SAMPLE PROCESSING, Westwood, MA (US)

(21) Appl. No.: 12/400,364

(22) Filed: Mar. 9, 2009

Related U.S. Application Data
(63) Continuation-in-part of application No. PCT/US2007/019639, filed on Sep. 10, 2007.
(60) Provisional application No. 60/843,347, filed on Sep. 8, 2006.
(60) Provisional application No. 60/843,176, filed on Sep. 8, 2006.
(60) Provisional application No. 60/861,993, filed on Nov. 30, 2006.

(51) Int. Cl. C12N 1/02; C12M 1/00

(52) U.S. Cl. 435/261; 435/308.1

(57) ABSTRACT

A centrifugal device and method are provided for the separation of buoyant material such as parasitic ova from fecal matter. A rotor assembly, rotatable about its central axis in a centrifuge, includes a housing with a centrally located top opening leading to a centrally located mixing chamber. An annular sediment chamber is provided, also coaxial about the central axis, connected by a passage with the mixing chamber. A coring assembly is used to retrieve and insert a fecal sample into the mixing chamber for mixing with a flotation fluid. During centrifugation, heavier fecal components pass radially outwardly to the sediment chamber while the ova collect on the inward surface of the flotation fluid. After centrifugation, more flotation fluid is added, if needed, until a meniscus forms at the top opening. A coverslip is placed over the top opening and the ova float to the surface of the fluid and adhere to the coverslip. The coverslip is removed and the ova detected using standard microscopy procedures. In another aspect, a centrifugal device is provided in which the ova are delivered through centrifugation to a pipette tip for dispensing onto a microscope slide or coverslip.

### CROSS REFERENCE TO RELATED APPLICATIONS

This application is a continuation-in-part of and claims the benefit under 35 U.S.C. § 120 of PCT International Application No. US2007/019639, filed Sep. 10, 2007, which claims priority under 35 U.S.C. § 119(e) of U.S. Provisional Patent Application No. 60/843,347, filed Sep. 8, 2006, U.S. Provisional Patent Application No. 60/843,176, filed Sep. 8, 2006, and U.S. Provisional Patent Application No. 60/861,993, filed Nov. 30, 2006, the disclosures of all of which are incorporated by reference herein.

### STATEMENT REGARDING FEDERALLY SPONSORED RESEARCH OR DEVELOPMENT

N/A

### BACKGROUND OF THE INVENTION

It has long been recognized that analyzing fecal specimens for parasite ova by microscopy is a simple and effective method for identifying parasites afflicting a patient. This method is routinely used in clinical and veterinary laboratories around the world to identify specific parasites in fecal specimens from animals and humans so that the patient may be properly treated for the affliction.

There are a variety of laboratory techniques in common use to detect the presence of ova in a fecal sample. The simplest of these is the direct smear technique in which a small sample of patient feces is mixed with saline and “smeared” across the surface of a microscope slide. A coverslip is placed over the smear and the specimen is examined microscopically for parasite ova. This technique is rarely used in modern laboratories because the presence of debris in the fecal sample makes direct examination extremely difficult and prone to error. Also, the small sample size used makes it likely that a low population of parasites, such as during the early stages of an infestation, may not be detected.

For many years the preferred technique, in several variations, has been the use of a float-or-sink process in which a reagent liquid of a density between that of the ova and that of other fecal matter is vigorously mixed into the fecal specimen to allow ova contained within the feces to be released to the liquid, and the ova then allowed to separate by floatation from the fecal debris. The ova, having floated to the top of the liquid, are then transferred to a microscope slide, such as by touching a coverslip to the surface of the liquid and placing the coverslip onto a microscope slide. Under the microscope, the type of ova and therefore the specific parasites present in the sample can be identified, and the seriousness of the infestation can be determined by counting and recording the number of each type of ova. This prior art process has been improved over the years, but still is not optimized and suffers from several limitations including the risk of exposure of laboratory personnel to potentially dangerous pathogens, complexity, unpleasant odor and also a degree of unreliability or inaccuracy.

Early improvements to this procedure were a) the prefiltration of the feces and floatation mixture, typically through a strainer, to remove clumps and undigested vegetable matter which may be contained in the fecal specimen and which would float to the surface of the liquid along with the ova, and b) the centrifugation of the prefiltered mixture to accelerate the process and provide a sharper separation of ova and fecal debris. While this latter technique proved to be more accurate and reliable, the requirement for a relatively large and costly centrifuge, the multiple transfer steps involved and the high potential for spills and aerosol generation limited the acceptance of the technique as a routine laboratory procedure.

From the 1970's onward, a number of low-cost stand-alone devices were introduced which combined sample collection, filtration of debris, and ova collection in a single disposable unit. The first of these devices, called the FECALYZER, consists of a container and a combination sample collection and straining part. In operation, the container is filled with a flotation reagent, the sample collector and strainer is used to collect a measured amount of feces and to effect mixing of the sample and flotation liquid in the container to release ova from the fecal sample and to allow passage of the ova through the integral strainer while retaining vegetable matter and other fecal debris in the lower portion of the device. A coverslip is placed upon the opening of the device at the liquid surface to receive the floating ova. While the FECALYZER and other similar devices such as the OVASSAY are widely used today, particularly in companion animal veterinary practices, the devices of this technique have several key deficiencies including a) the time required for complete floatation of ova to the surface of the liquid can be excessive thereby making it impossible to provide a diagnosis during a typical one-half hour patient appointment and b) without the benefit of prefiltration or pre-separation by centrifugation, the straining method of these devices under natural gravity may trap a portion of the ova thereby contributing to inaccuracy of the diagnosis.

The accuracy and sensitivity of veterinary fecal exams have recently come under close scrutiny because many common parasites found in companion animals can be transmitted from pets to owners. As a result, a number of professional veterinary societies have studied the accuracy and repeatability of various techniques for fecal parasite analysis and have universally concluded that only the centrifugal floatation method can produce the accuracy and sensitivity necessary to protect both pets and their owners and that the widely-used FECALYZER and similar devices are suboptimal for the procedure.

### SUMMARY OF THE INVENTION

A centrifugal fecal analyzer device and centrifugal floatation method are provided for the separation of ova from human and animal fecal specimens and the subsequent collection of ova onto a microscope slide for microscopic examination. The device and method are simple, rapid and highly accurate and can be used on a routine basis in veterinary and clinical laboratories. The device and method overcome the limitations and problems of the prior art noted above.

More specifically, the centrifugal fecal analyzer device and method provide increased test efficiency and improved accuracy. The device and method allow rapid separation of ova from fecal matter and allow testing and diagnosis within a typical one-half hour patient appointment. The number of steps required in the analysis are limited, and only minimal handling of feces is required, allowing collection of parasitic ova without significant contamination of laboratory personnel, equipment or facilities. Collection of parasite ova can be accomplished with minimal interference from fecal debris and other buoyant material that may be contained in the fecal sample, such as vegetation.

More particularly, a centrifugal device for separation of buoyant material includes a rotor assembly and a coring assembly to allow collection and addition of a fecal sample to the rotor assembly. The rotor assembly has a housing coaxial and rotatable about a central axis. The housing includes at least a top element and a bottom element, with an opening centrally located in the top element. A mixing chamber is centrally located within the housing and coaxial about the central axis. An annular sediment chamber is coaxially disposed within the housing radially outwardly of the mixing chamber, with a passage between the mixing chamber and the sediment chamber to allow heavier fecal components to pass radially outwardly during a centrifuge spin cycle.

A coring assembly is used to collect a fecal sample and insert it into the rotor assembly through the central top opening. The coring assembly includes a lower portion having a coring element and optionally a filter element and a removable upper portion or handle.

In operation, a user adds flotation fluid into the rotor assembly. The user cores a fecal sample with the coring assembly and inserts the coring assembly into the opening in the rotor assembly. The user rotates and/or reciprocates the coring assembly to break up the sample and mix it with the flotation fluid. Optionally, the user may cap the device and shake it to further mix the sample. The user removes the detachable handle from the coring assembly, leaving the coring element in the rotor assembly. Sufficient fluid may be added at this stage to completely fill the rotor assembly. The rotor assembly is placed in a centrifuge and spun for an appropriate time, during which fecal matter and debris that are more dense than the flotation fluid are forced outwardly from the center of rotation into the sediment chamber, while the less dense ova are forced inwardly to the surface of the liquid in the mixing chamber. After centrifugation, the rotor assembly is removed from the centrifuge and placed on a level surface, and more flotation fluid is added, if necessary, through the central opening, or through a fill port if present, until a meniscus is created at the opening of the rotor assembly. A coverslip is placed over the rotor assembly opening and after a short period of time, as needed, the ova rise up in the flotation fluid and adhere to the coverslip. The coverslip can then be removed and transferred to a microscope slide for analysis using standard microscopy methods.

In another aspect of the present invention, a centrifugal device is provided in which the ova are delivered through centrifugation to a pipette tip for dispensing onto a microscope slide or coverslip. In this manner, the wait for ova to rise through a flotation fluid is also minimized.

### DESCRIPTION OF THE DRAWINGS

The invention will be more fully understood from the following detailed description taken in conjunction with the accompanying drawings in which:

FIG. 1 is a cutaway isometric view of a first embodiment of a centrifugal device rotor assembly according to the present invention;

FIG. 2 is an exploded isometric view of the rotor assembly of FIG. 1 and a coring assembly according to the present invention;

FIG. 3 is an isometric view of the coring assembly inserted in the rotor assembly of FIG. 2;

FIG. 4 is an isometric view illustrating a handle of the coring assembly removed from a coring element;

FIG. 5 is an isometric view illustrating filling the rotor assembly with a flotation fluid;

FIG. 6 is an isometric view illustrating a coverslip placed over the rotor assembly for collection of ova thereon;

FIG. 7 is an isometric view of the coring assembly of FIG. 2;

FIG. 8 is an isometric view of the coring element of the coring assembly;

FIG. 9 is a cutaway view of the coring element of FIG. 8;

FIG. 10 is a schematic plan view illustrating the mixing of a fecal sample;

FIG. 11 is a further embodiment of a coring assembly;

FIG. 12 is a still further embodiment of a coring assembly;

FIG. 13 is an exploded, partially cutaway isometric view of a second embodiment of a centrifugal device with a rotor assembly and coring assembly;

FIG. 14 is an exploded cross-sectional view of the rotor assembly of FIG. 13;

FIG. 15 is a cross-sectional side view of the assembled rotor assembly of FIG. 13;

FIG. 16 illustrates the insertion of a fecal sample in the rotor assembly of FIG. 13;

FIG. 17 illustrates mixing of the fecal sample with flotation fluid in the mixing chamber of the rotor assembly of FIG. 13;

FIG. 18 illustrates seating of the coring element in the rotor assembly of FIG. 13 and removal of the handle of the coring assembly;

FIG. 19 illustrates a centrifuge spin cycle of the rotor assembly of FIG. 13;

FIG. 20 illustrates addition of flotation fluid to form a meniscus in the top opening of the rotor assembly of FIG. 13;

FIG. 21 illustrates ova adhering to a coverslip placed over the top opening of the rotor assembly of FIG. 13;

FIG. 22 illustrates removing the coverslip with ova adhering thereto;

FIG. 23 is a cross-sectional side view of a still further embodiment of a centrifugal device rotor assembly and coring assembly according to the invention;

FIG. 24 is an exploded view of the rotor assembly and coring assembly of FIG. 23;

FIG. 25 is an exploded view of a still further embodiment of a rotor assembly of a centrifugal device;

FIG. 26 illustrates filling the rotor assembly of FIG. 25 with a fecal sample through a syringe filter to provide prefiltration of the fecal sample;

FIG. 27 illustration a centrifuge spin cycle of the rotor assembly of FIG. 25;

FIG. 28 illustrates deposition of ova in a collection trough of the rotor assembly of FIG. 25;

FIG. 29 illustrates addition of flotation fluid to form a meniscus in the top opening of the rotor assembly of FIG. 25;

FIG. 30 illustrates ova adhering to a coverslip placed over the top opening of the rotor assembly of FIG. 25;

FIG. 31 is a still further embodiment illustrating the use of an optional screen with the rotor assembly of FIG. 25;

FIG. 32 illustrates blockage of larger clumps by the screen of FIG. 31;

FIG. 33 is a cross-sectional side view of a further centrifugal device according to the present invention;

FIG. 34 is an exploded view of the centrifugal device of FIG. 33;

FIG. 35 illustrates placing a fecal sample into the container of the device of FIG. 33;

FIG. 36 illustrates adding a flotation fluid to the container of the device of FIG. 33;

FIG. 37 illustrates mixing the fecal sample in the flotation fluid of the device of FIG. 33;

FIG. 38 illustrates closing the container of the device of FIG. 33;

FIG. 39 illustrates inverting the container and removing the plug of the device of FIG. 33;

FIG. 40 illustrates attaching the centrifuge assembly to the container of the device of FIG. 33;

FIG. 41 illustrates transferring the fecal sample in the flotation fluid to the centrifuge assembly of the device of FIG. 33;

FIG. 42 illustrates transferring the fecal sample and fluid until the centrifuge assembly is completely filled;

FIG. 43 illustrates removing the centrifuge assembly from the container of the device of FIG. 33;

FIG. 44 illustrates placing the centrifuge assembly in a centrifuge along with a balance tube;

FIG. 45 illustrates centrifugation of the centrifuge assembly, with the ova forced inwardly to the outlet of the device of FIG. 33;

FIG. 46 illustrates removing the centrifuge assembly from the centrifuge;

FIG. 47 illustrates placing a few drops of the ova in the flotation fluid onto a slide; and

FIG. 48 illustrates a further embodiment of a rotor assembly incorporating dispersion elements to facilitate the break up of fecal matter.

### DETAILED DESCRIPTION OF THE INVENTION

The disclosures of PCT International Application No. US2007/019639, filed Sep. 10, 2007, and U.S. Provisional Patent Application No. 60/843,347, filed Sep. 8, 2006, U.S. Provisional Patent Application No. 60/843,176, filed Sep. 8, 2006, and U.S. Provisional Patent Application No. 60/861,993, filed Nov. 30, 2006, are incorporated by reference herein.

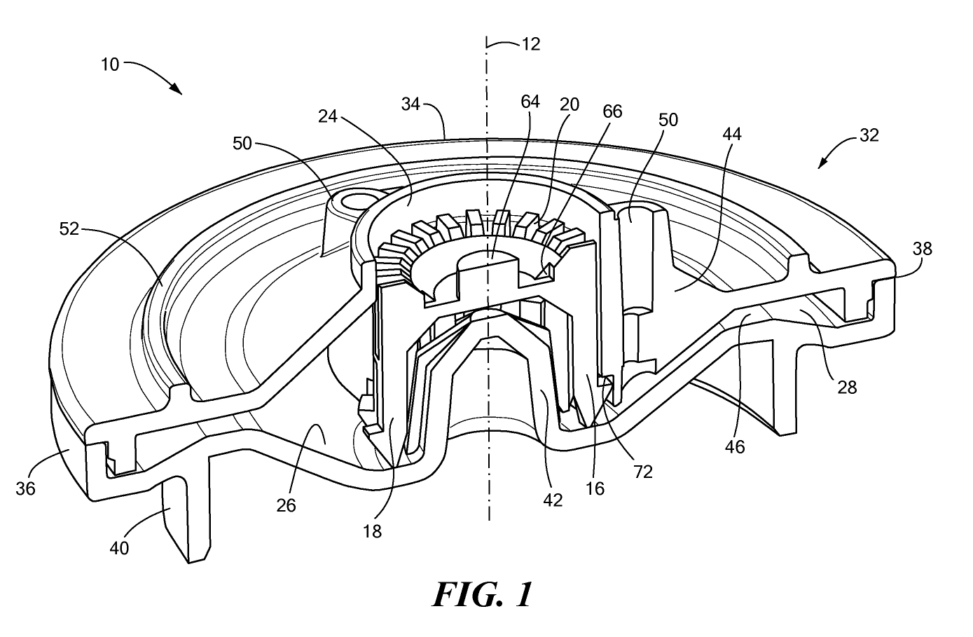

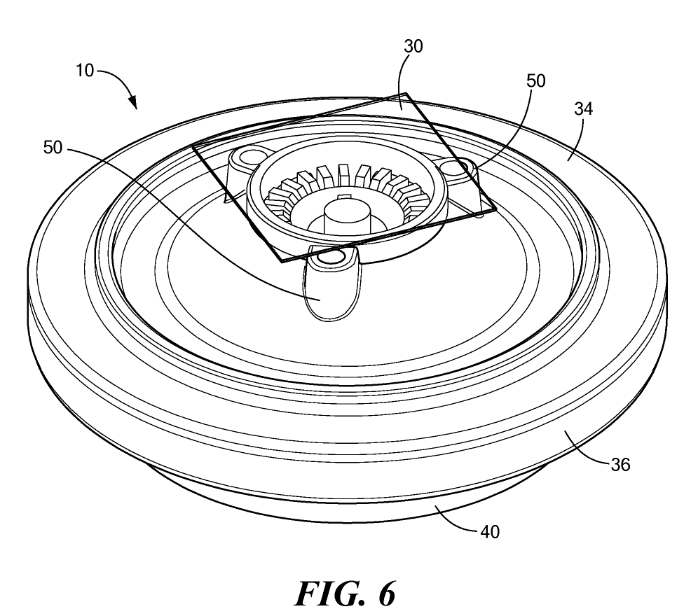

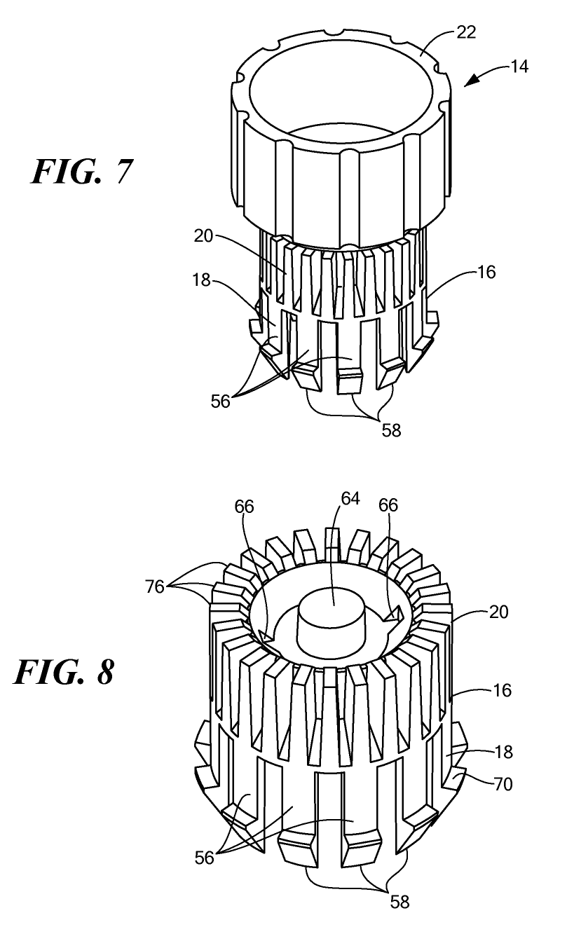

A first embodiment of a centrifugal device is illustrated in FIGS. 1-10. The device includes a rotor assembly 10 for rotation about its central axis 12 in a centrifuge (not shown) and a coring assembly 14. The coring assembly includes a lower portion 16 having a coring element 18 and optionally a filter element 20, and a removable upper portion or handle 22. The coring assembly is used to retrieve a fecal sample and insert it into the rotor assembly through an opening 24 in the top. The rotor assembly includes a mixing or receiving chamber 26, in which the fecal sample is initially mixed with a flotation fluid, and a sediment or separation chamber 28, in which the heavier components of the fecal sample collect during centrifugation. After centrifugation, any ova in the fecal sample remain in the mixing chamber 26, and more flotation fluid is added if necessary to the mixing chamber until a meniscus is formed at the top opening 24. A coverslip 30 is placed over the top opening (FIG. 6). The ova contained in the fluid layer float to the top and adhere to the coverslip. The filter element of the coring assembly prevents any unwanted floating material from reaching the coverslip as the fluid level is raised, while ova are able to pass through to the coverslip. The coverslip is removed and the ova thereon detected using standard microscopy methods.

The rotor assembly 10 includes a housing 32 that is generally annular and circular in plan view and coaxial about the central axis 12 so that it can be rotated about that axis in a centrifuge. In the embodiment illustrated, the rotor assembly housing includes a top element 34 and a bottom element 36 joined and sealed about an annular periphery 38, such as by ultrasonic welding or solvent bonding. An annular rib 40 forming a rotor holder element depends from the bottom element 36 for mounting in a centrifuge. See for example, U.S. Pat. No. 4,846,974. The rotor assembly can be configured for use with other centrifuges, as will be appreciated by those of skill in the art.

The opening 24 is centrally located in the top element 34, coaxial with the central axis 12. A mixing post or bayonette 42 extends upwardly from the bottom element 36 below the opening 24 and coaxial with the central axis 12. The mixing post preferably includes ribs or other irregularities disposed coaxially about the central axis, which assist in the breaking up of the fecal sample during mixing. The mixing post is preferably molded integrally or unitarily with the bottom element, although it could also be formed as a separate piece if desired.

The mixing chamber 26 is annularly disposed coaxially about the central axis 12 and surrounding the mixing post 42. In the region of the mixing chamber, the top and bottom elements 34, 36 taper radially toward each other in the direction of the periphery 38. Inner surfaces of the top and bottom elements form the walls of the mixing chamber. One or more vanes 44 may be disposed in the mixing chamber to minimize turbulence and mixing generated in the chamber upon deceleration of the centrifuge. The vanes are formed by narrow radial wall portions. In the embodiment shown, the vanes are integrally formed with the top element extending down to a location close to the bottom element. Alternatively, the vanes could be formed integrally with the bottom element, extending up toward the top element.

An annular capillary gap 46 forms a passage between the mixing chamber 26 and the sediment chamber 28. The gap is defined by a narrow space between the inner surfaces of the top and bottom elements radially outward of the mixing chamber and coaxial with the central axis. The narrow size of the capillary gap minimizes the transfer of turbulence from the sediment chamber to the mixing chamber during deceleration.

The sediment chamber 28 is annularly disposed between the top and bottom elements 34, 36 outwardly of the capillary gap 46 and coaxial about the central axis 12. At the sediment chamber, the inner surface of the bottom element is spaced below the level of the capillary gap to aid in trapping the fecal components within the sediment chamber.

One or more fill ports 50 are optionally and preferably formed in the top element 34 of the rotor assembly 10. Each fill port provides a passage for the introduction of fluid into the mixing chamber 26 from outside the rotor assembly. The fill port is formed by a passage extending from an entrance opening in the top surface of the top element to an exit opening near the bottom of the mixing chamber. Preferably, the fill port is molded integrally or unitarily with the top element of the rotor assembly. Optionally, each fill port can be associated with a vane 46 to provide additional structural support for the vane and/or for ease of molding.

After centrifuging, fluid is introduced if necessary through the fill port 50 into the mixing chamber 26 to raise the fluid level in the mixing chamber until it forms a meniscus at the top opening 24 of the rotor assembly. An annular lip 52 may be formed on the outer surface of the top element 34 to capture any fluid that spills over the opening 24 during filling. Introducing fluid near the bottom of the mixing chamber 26 allows the fluid level to be raised without disrupting the fluid surface. By minimizing disruption of the fluid surface, the ova do not become remixed into the fluid, but remain near the top of the fluid.

The coring element 16 of the coring assembly 14 includes a number of fingers 56 arranged in a cylindrical configuration that are pressed or cored into fecal material for collection of a fecal sample. The fingers preferably have tapered ends or tips 58, which help the fingers core into the fecal sample. When the coring assembly is removed from the fecal sample, a portion of the fecal sample is retained within the fingers.

To assist in breaking up the sample, a small amount of flotation fluid is added to the mixing chamber 26 through the top opening 24, generally before the fecal sample is added. When the coring assembly 14 is inserted through the opening in the top of the rotor assembly, the coring element 16 fits over the mixing post 42. The fecal sample 60 retained within the coring element is broken up by being pushed radially outwardly through the fingers 56 of the coring element by the mixing post. See FIG. 10. A user rotates and reciprocates the coring element by the handle, thereby breaking up the fecal sample further.

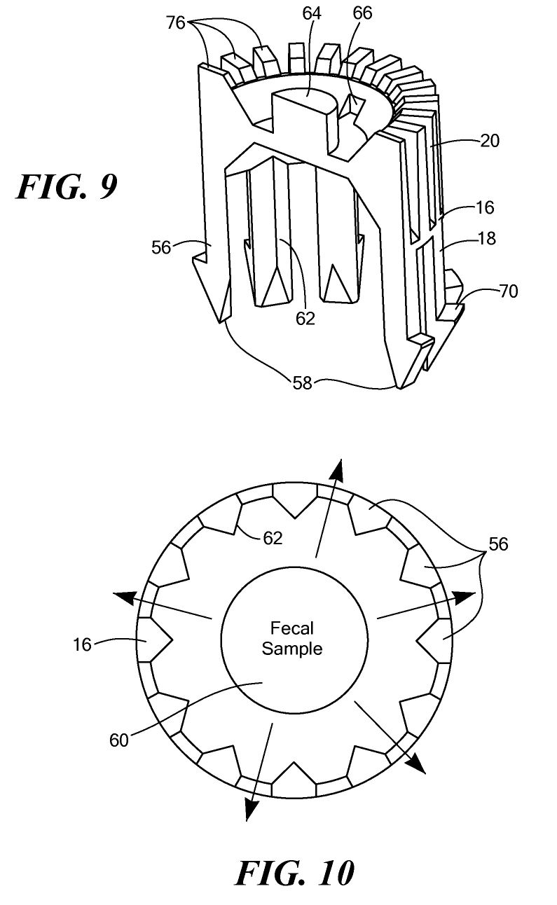

Each finger 56 is also preferably shaped as a wedge 62 in cross section, best illustrated in FIGS. 9 and 10. The wedge shape prevents material from catching on inwardly-facing surfaces of the fingers, allowing more of the sample to pass radially through the fingers into the mixing chamber. This leads to a more complete sample analysis and concomitantly a greater likelihood of detecting ova if present.

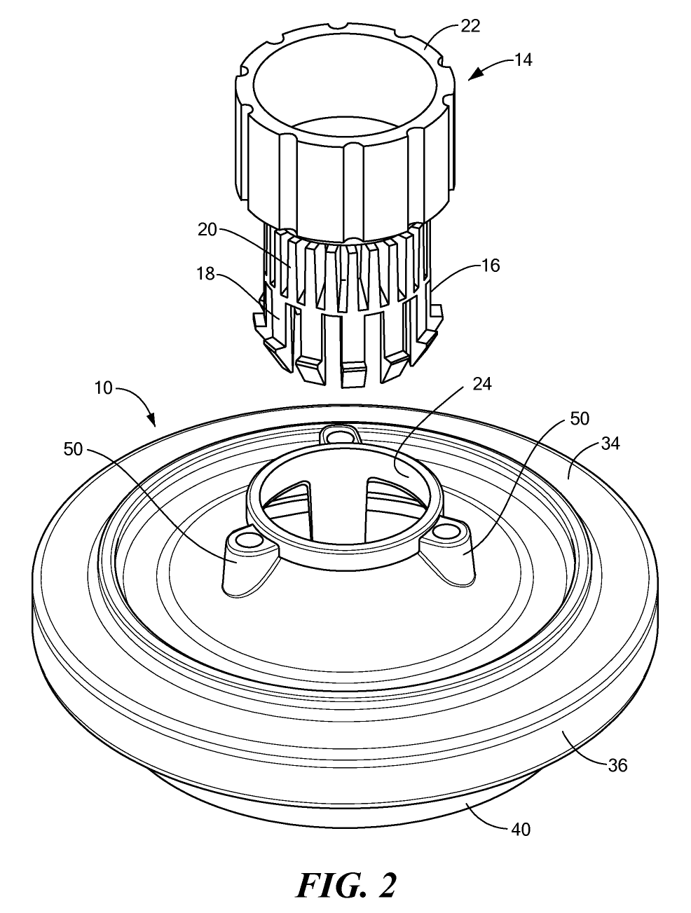

As noted above, the coring assembly 14 also includes a handle 22 extending upwardly from the coring element 18. A user grasps the coring assembly by the handle for inserting the coring element into the fecal material and into the rotor assembly. The user also rotates the coring assembly by rotating the handle. The handle illustrated in FIGS. 2-4 and 7 includes a cylinder of a diameter to be gripped between the thumb and finger of a user's hand. The cylinder is preferably knurled or ribbed for further ease of gripping.

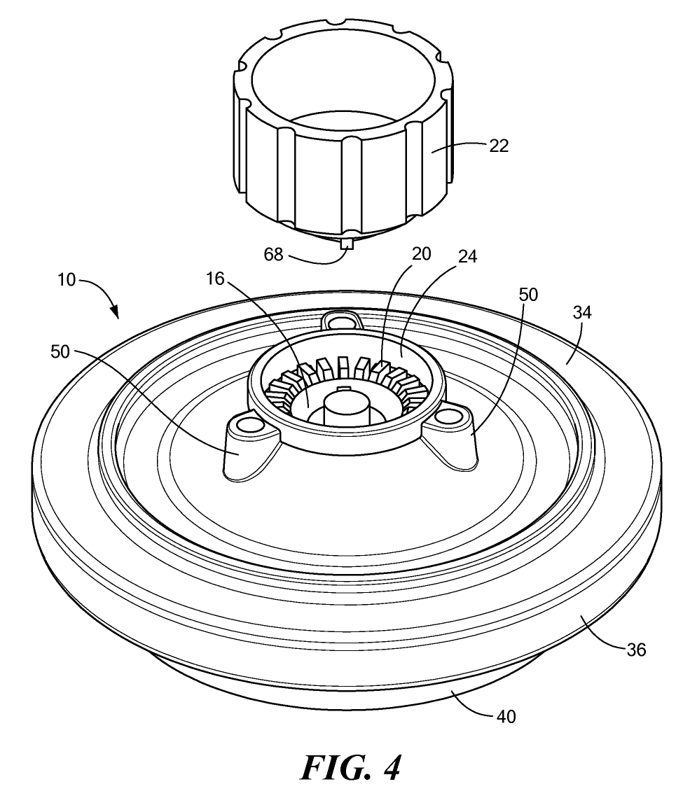

The handle 22 is removably attached to the coring element 18. When the handle is removed, the coverslip 30 can be placed over the opening 24 after centrifugation. In the embodiment illustrated in FIGS. 1-9, the coring element includes a hub 64 and two opposed notches 66. The handle includes a recess (not shown) that frictionally fits over the hub and two tabs 68 (see FIG. 4) that fit within the notches, thereby rotationally fixing the handle to the coring element. To remove the handle, the user pulls upwardly on the handle with sufficient force to remove it from the coring element. The handle can be removed after mixing and prior to centrifugation or after centrifugation, in which case the handle can serve as a cap during spinning.

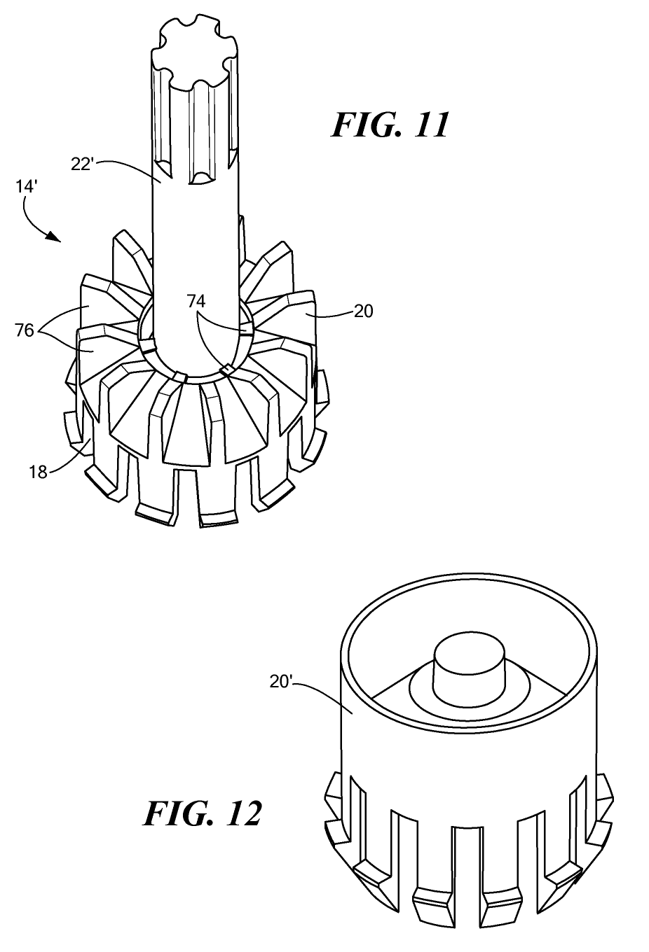

In an alternative embodiment illustrated in FIG. 11, a coring assembly 14′ includes a handle 22′ that is attached to the coring element with breakaway tabs 74. After inserting the coring assembly into the housing, the user bends the handle to break the tabs. In a further alternative, the handle can be attached with a snap fit. The handle can be made removable in any other manner, as will also be appreciated by those of skill in the art.

Preferably, the coring element 18 includes a locking mechanism to ensure that the coring element is retained within the rotor assembly 10 when the handle is removed. In the embodiment shown in FIGS. 1-9, the locking mechanism is provided by an upwardly facing shoulder 70 on each finger 56 that mates with a corresponding downwardly facing surface or surfaces 72 formed in the top element 34. For example, the downwardly facing surfaces 72 may be formed at each fill port 50. When the coring element is inserted through the opening in the rotor assembly, some of the shoulders 70 snap into place under the downwardly facing surfaces 72. Other locking mechanism configurations may be provided, as will be appreciated by those of skill in the art.

As noted above, the lower portion 16 of the coring assembly 14 also optionally and preferably contains a filter element 20 above the coring element that mates within the opening 24. In cooperation with the opening 24, the filter element 20 limits the passage of heavier and larger fecal components such as undigested vegetable matter and large debris such as kitty litter back toward the central axis and up to the surface of the fluid with the ova. In the embodiments of FIGS. 1-11, the filter element is formed by a number of closely spaced fins or ribs 76. In other embodiments, illustrated schematically in FIG. 12, the filter element 20′ can be formed, for example, of a woven or molded plastic screen, a metal screen, or porous plastic. The filter elements 20, 20′ eliminate the need for a separate prefiltration step, simplifying the process and shortening the time to perform the test.

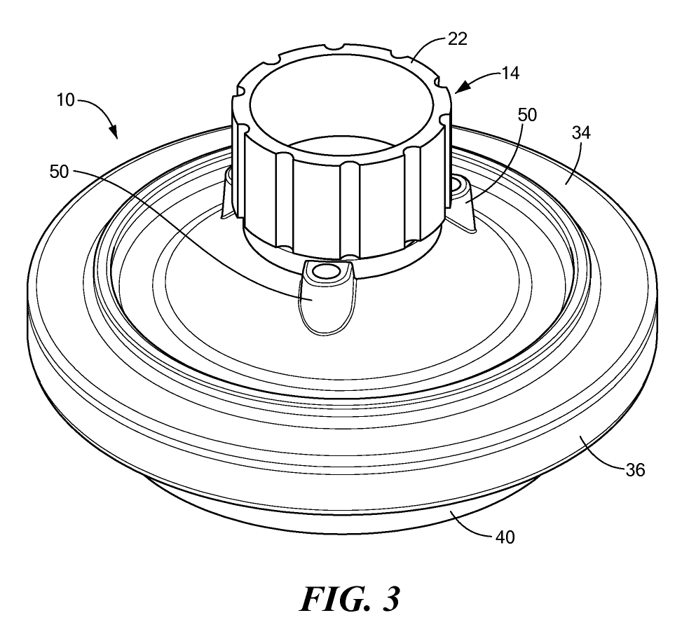

In operation, a user adds a quantity of flotation fluid (e.g., 3 to 7 ml, although the quantity depends on the size of the rotor assembly) into the rotor assembly. A fluid such as zinc sulfate with a specific gravity of 1.18 is suitable for separation of parasitic ova; other suitable flotation fluids are known in the art. If a fill line is present as an aid, the user may add fluid until it reaches the fill line. The user cores a fecal sample with the coring assembly and inserts the coring assembly into the opening in the top of the rotor assembly. See FIGS. 2 and 3. The user rotates the coring assembly back and forth to break up the sample and mix it with the flotation fluid. If desired, sufficient flotation fluid can be added at this stage to completely fill the rotor assembly, obviating the need to add more fluid later and wait for the ova to rise to the surface.

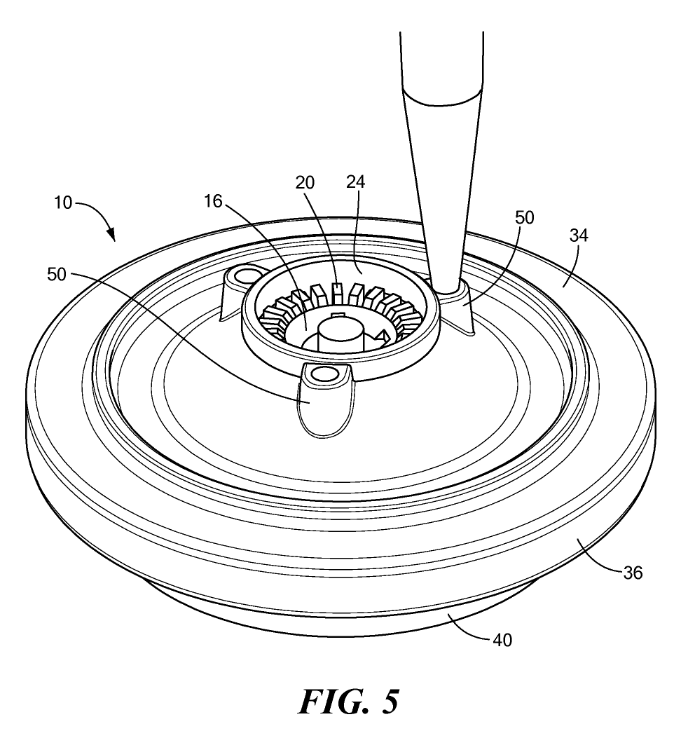

The user removes the detachable handle for the coring assembly. See FIG. 4. The filter element is retained in the rotor. If necessary, the rotor assembly is capped at the opening, particularly if the rotor is completely filled with flotation fluid. The rotor assembly is placed in a centrifuge and spun for an appropriate time, for example, 90 seconds. As the spin continues, fecal matter and debris that are more dense than the flotation fluid are forced outwardly from the center of rotation into the sediment chamber, while the less dense ova are forced inwardly to the surface of the liquid in the mixing chamber. Upon completion of the spin cycle and deceleration to a stop, the user removes the rotor assembly from the centrifuge, places it on a level surface, and adds more flotation fluid through one of the fill ports until a meniscus is created at the opening of the rotor assembly. See FIG. 5. With a fill port that extends to the bottom of the rotor assembly, the fluid level is raised without disrupting the fluid surface. By minimizing disruption of the fluid surface, the ova do not become remixed into the fluid, but remain near the top of the fluid.

Alternatively, if the rotor assembly has previously been completely filled, this step may be eliminated, or only a few drops of fluid may need to be added to achieve a meniscus at the opening. The meniscus at the opening may be achieved in an other manner, for example, by an increase in pressure in the rotor assembly.

The user then places a coverslip over the rotor assembly opening and waits a short period of time, if necessary, (for example, 1 to 2 minutes if the ova need to rise to the surface) before removing the coverslip. See FIG. 6. The ova are then contained in the fluid layer that adheres to the coverslip and, once the coverslip is placed onto a microscope slide, can be detected using standard microscopy methods. The process can typically be performed in about 5 minutes, compared to about 30 minutes for prior art processes. The rotor assembly and coring assembly are typically discarded after use for sanitary reasons. If the handle is replaceable, as in the embodiment of FIGS. 1-10, the handle can be replaced on the rotor assembly to seal the contents within the rotor assembly for discarding.

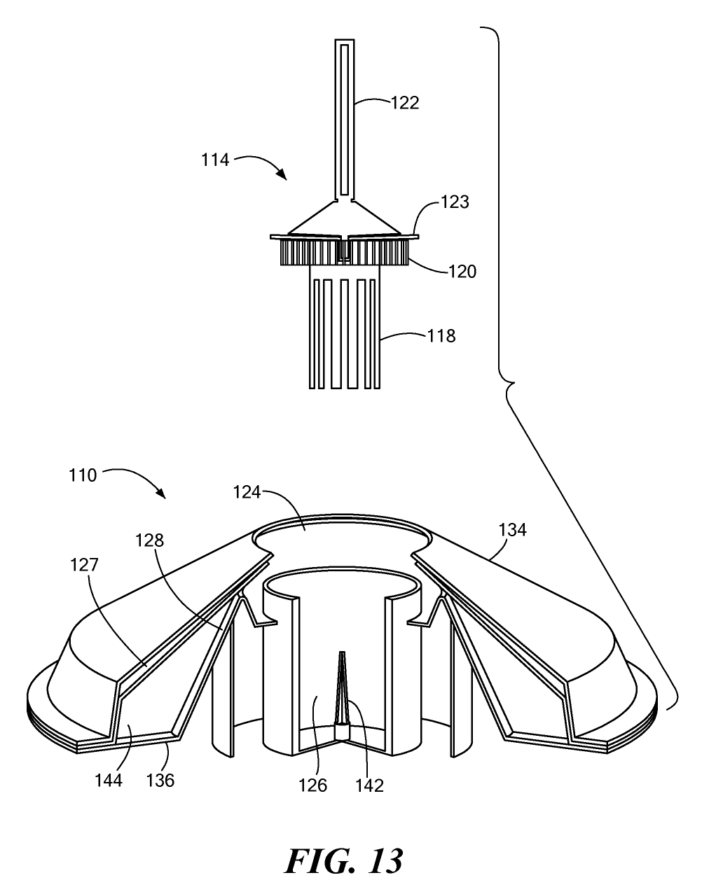

A further embodiment of a centrifugal device is illustrated in FIGS. 13-22. As with the previously described embodiment, the rotor assembly 110 is generally annular and circular in plan view and coaxial about a central axis 112. The rotor assembly is formed with a housing in three parts: a top element 134, a middle element 135, and a bottom element 136. A central opening 124 is formed through the top and middle elements. An overspillage lip 152 may be formed on an upper surface of the top element.

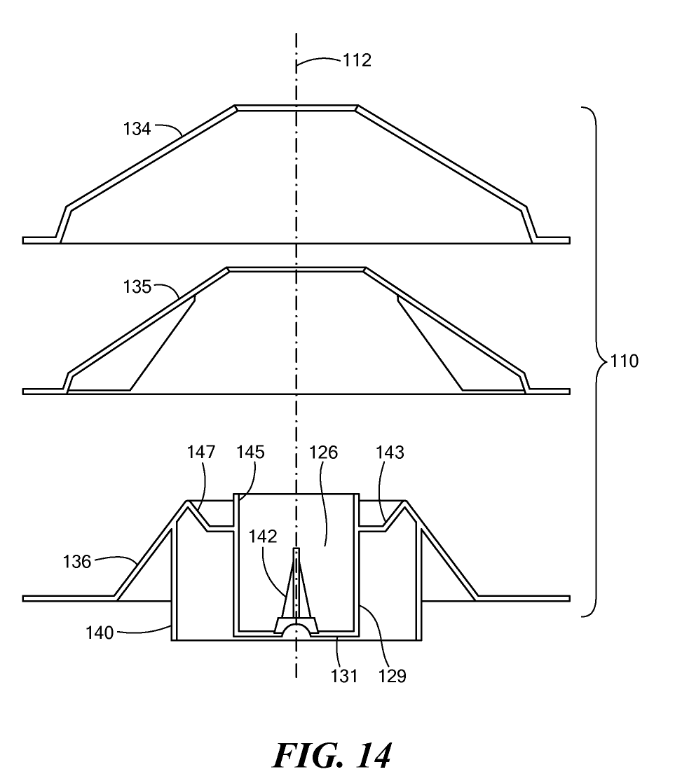

An overflow chamber 127 is formed between the top element 134 and the middle element 136. A sediment or separation chamber 128 is formed between the middle element 135 and the bottom element 136. A mixing chamber 126, which in conjunction with a coring assembly 114 also provides prefiltering of the fecal sample, is formed in the bottom element 136 by a cylindrical wall 129 and a bottom surface 131. A mixing post 142 extends upwardly from the bottom surface of the mixing chamber. An annular ova collection trough 143 coaxially surrounds the mixing chamber, formed by the upper end 145 of the cylindrical wall 129 of the mixing chamber and an upwardly angled wall 147 of the bottom element (FIG. 14). An annular rib 140 forming a rotor holder for mounting in a centrifuge depends from the underside of the bottom element.

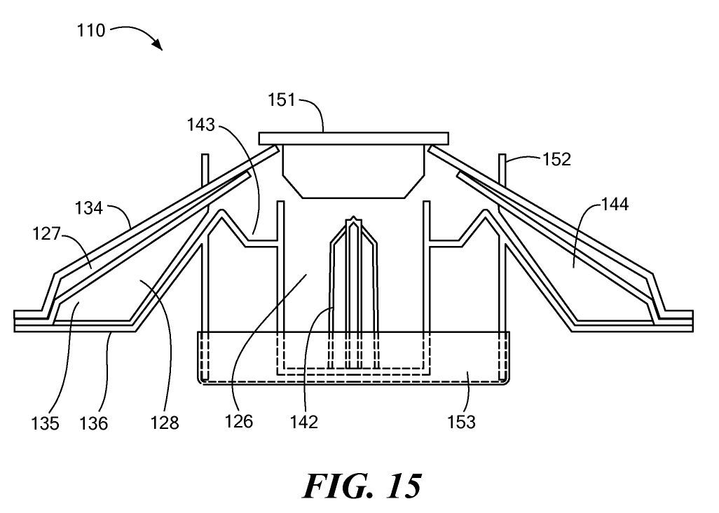

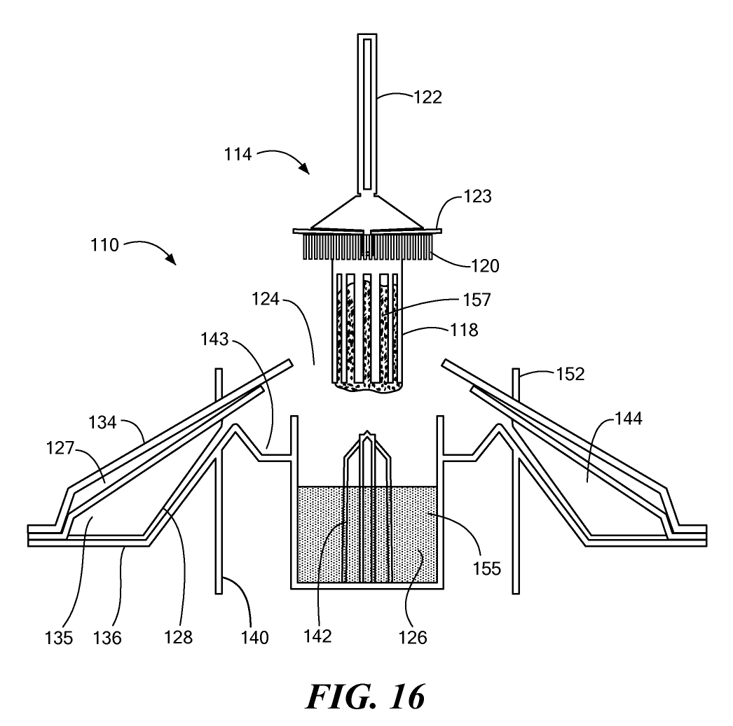

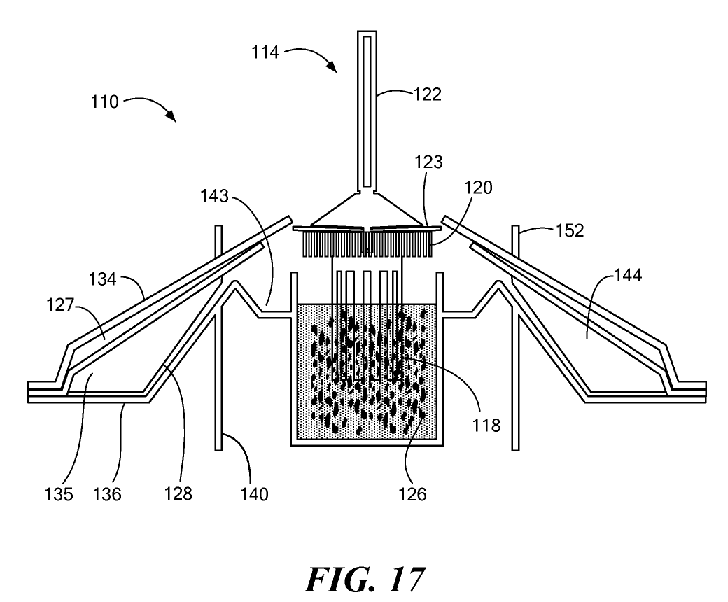

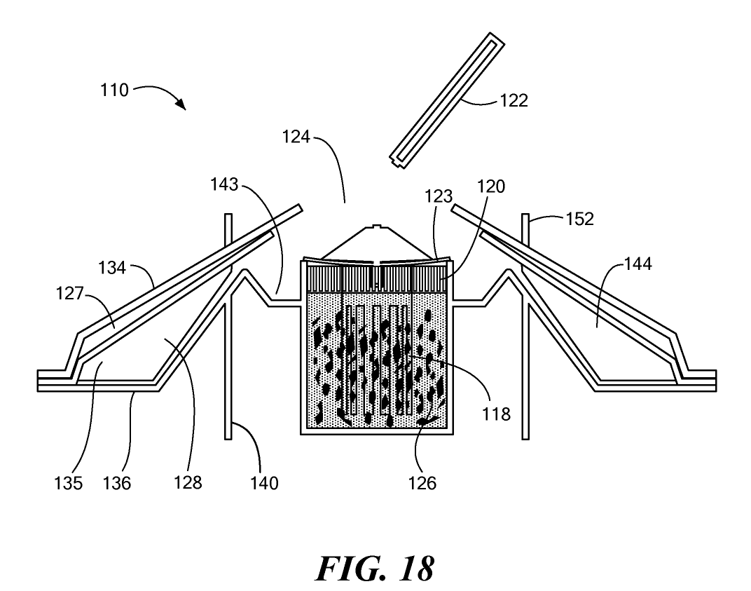

The rotor assembly 110 may be supplied with a top cap 151 over the central opening 124 and a bottom cap 153 over the rotor holder 140 (FIG. 15). In operation, the caps are removed and may be set aside for later use. A suitable amount of flotation solution 155 is added to the mixing chamber 126 (FIG. 16). The coring assembly 114 with a sample of fecal material 157 retained in a coring element 118 is inserted through the opening 124 into the mixing chamber 126 over the mixing post 142 (FIG. 17). The sample is mixed with the flotation solution by an up and down and swirling motion of the coring assembly. After the sample is fully mixed with the flotation solution, the coring assembly is pushed downwardly until a prefilter grid 120 seats within the opening of the mixing chamber 126. The handle 122 is then removed (FIG. 18), as discussed above. A one-way flapper valve 123 on the coring element seats against the top of the mixing chamber (FIG. 18).

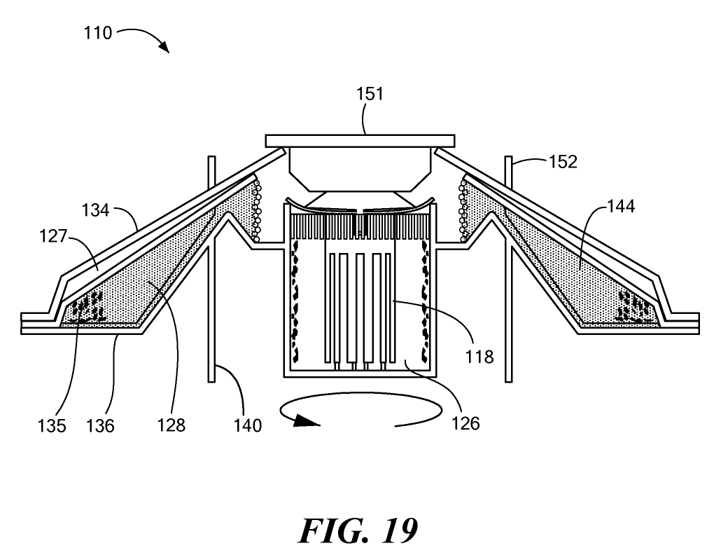

The top cap 151 is retrieved and placed over the rotor assembly opening 124, and the rotor assembly 110 is placed in a centrifuge (not shown). Upon acceleration of the rotor assembly, sample contained within the mixing chamber 126 is forced through the prefilter grid 120, past the one-way flapper valve 123, and into the sediment chamber 128 (FIG. 19). Larger components are retained by the prefilter grid in the mixing chamber, eliminating the need for a separate prefiltration step (FIG. 19).

Generally within one or two seconds, all of the fluid contained in the mixing chamber 126 is forced through the prefilter grid 120 and fills the sediment chamber 128. Any excess fluid overflows the top opening of the middle element and into the overflow chamber 127 through tiny weep holes or grooves along the top of the opening of the middle part.

As the spin continues, fecal matter and debris that are more dense than the flotation fluid are forced outwardly from the center of rotation, while the less dense ova are forced inwardly along the lower wall of the sediment chamber to the surface of the liquid contained within the collection trough 143 (FIG. 19). Upon completion of the spin cycle, typically about 90 seconds, the rotor is decelerated to a stop. Vanes or fins 144 contained within the sediment chamber 128 inhibit remixing. The ova remain suspended in the small amount of liquid contained in the collection trough 143. The rotor assembly is removed from the centrifuge, and the cap 151 is removed from the rotor assembly.

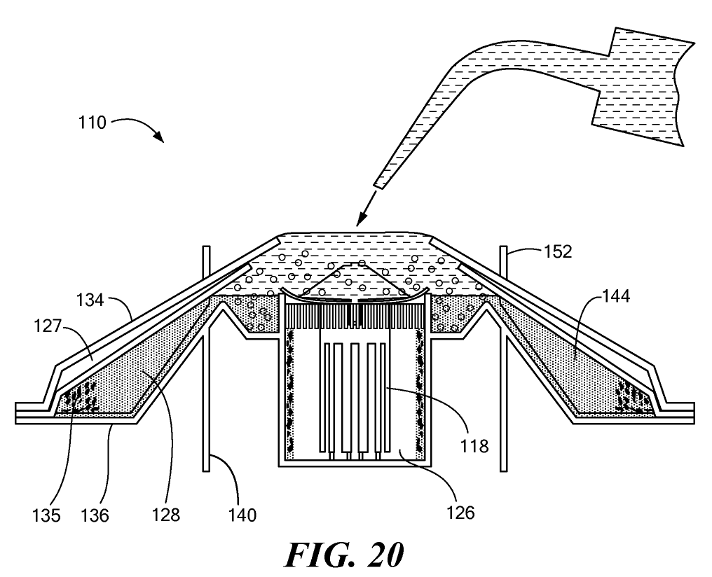

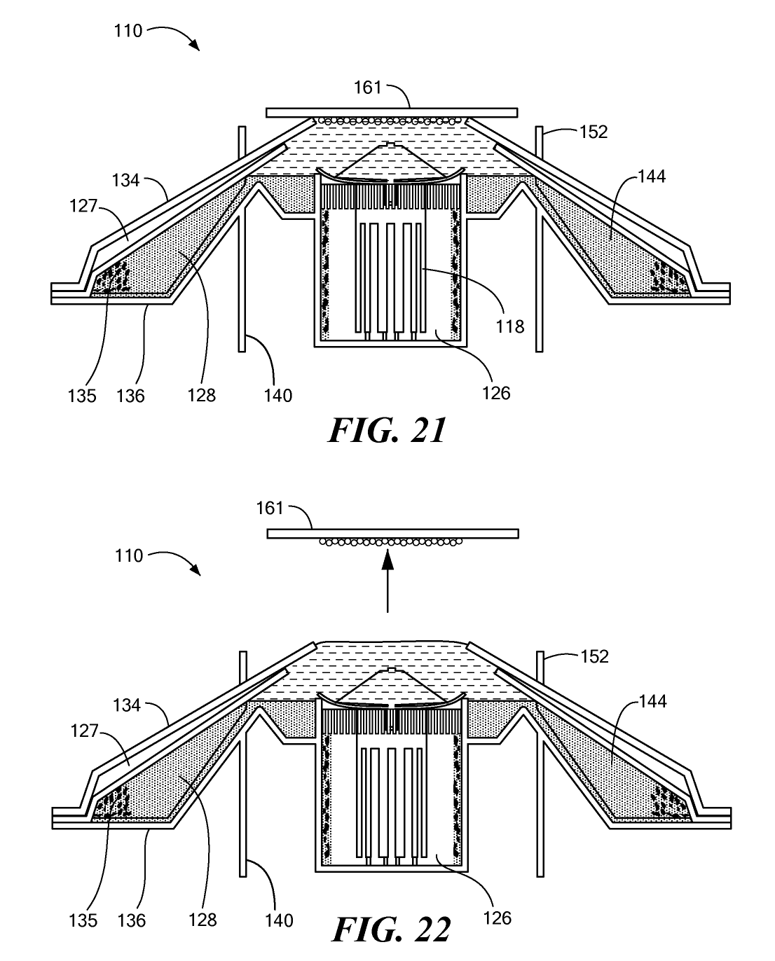

Flotation solution is added to the rotor assembly through the top opening 124 as necessary until a meniscus forms at the opening of the rotor assembly 110 (FIG. 20). Spillage is collected by the overspill lip 152 on the upper surface. The one-way flapper valve 123 covering the opening of the mixing chamber prevents fluid from entering the mixing chamber 126. The small weep holes at the opening of the overflow chamber prevent fluid from entering the overflow chamber under unit gravity while allowing fluid passage under centrifugal force. A coverslip 161 is placed over the top opening 124 of the rotor assembly 110. The rotor assembly is allowed to sit, for example, for 1-2 minutes, and the ova float to the underside of the coverslip. The coverslip is lifted upwardly off the rotor assembly, and ova contained within the fluid adhere to the underside of the coverslip (FIGS. 21, 22). The bottom cap may be placed over the overspill lip of the rotor assembly to seal the contents inside for discarding the rotor assembly in a sanitary manner.

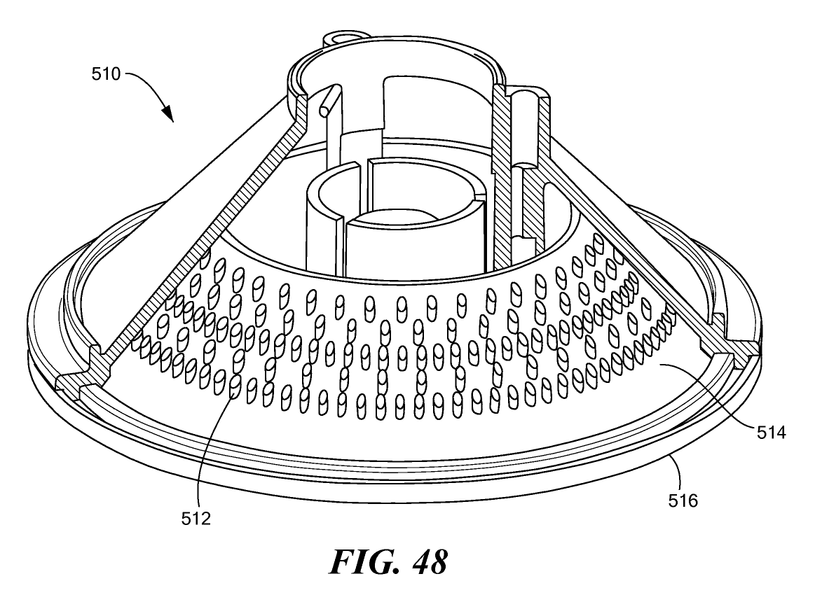

In another aspect of a rotor assembly, a dispersion element or elements can be included in the separation chamber. In the embodiment of the rotor assembly 510 illustrated in FIG. 48, the dispersion elements are formed as several rows of pins 512 in the separation chamber 514, upstanding from the bottom element 516. Other configurations can be provided, such as angled walls, dowels, or blades. The elements can be molded into the rotor base or added via a secondary manufacturing operation.

The dispersion elements facilitate the break up of fecal debris during the centrifugation. As the material is spun from the center of the rotor, it impacts the first row of pins and is then extruded through the openings between the pins. It then impacts another row of pins. Any desired number of rows and pattern of pins can be provided. For example, the pins can be arranged such that the material in passing through a row of pins encounters further pins centered on the flow path. Further, the passage between each row of pins can get progressively more narrow to facilitate the break up of the material. The size and shape of the pins can be adjusted and optimized for a particular consistency of material. Improved dispersion of the fecal matter increases the number of ova released into the flotation fluid, which improves the recovery efficiency of the device.

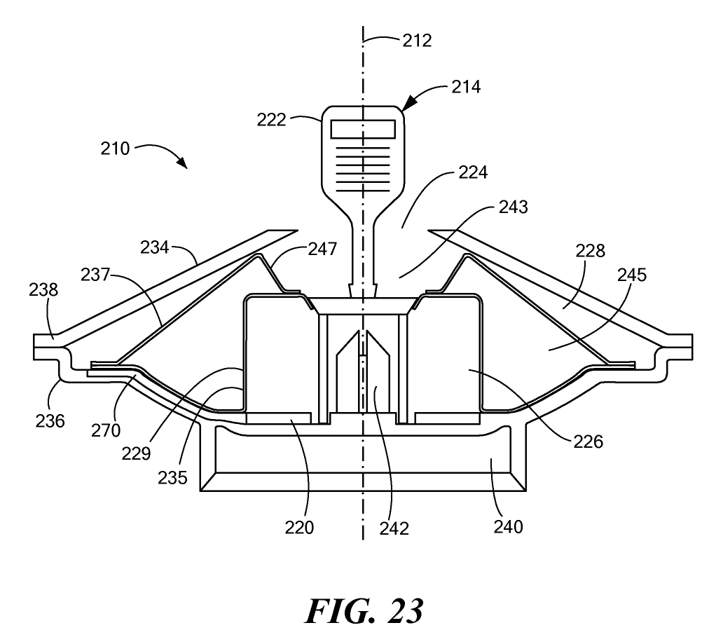

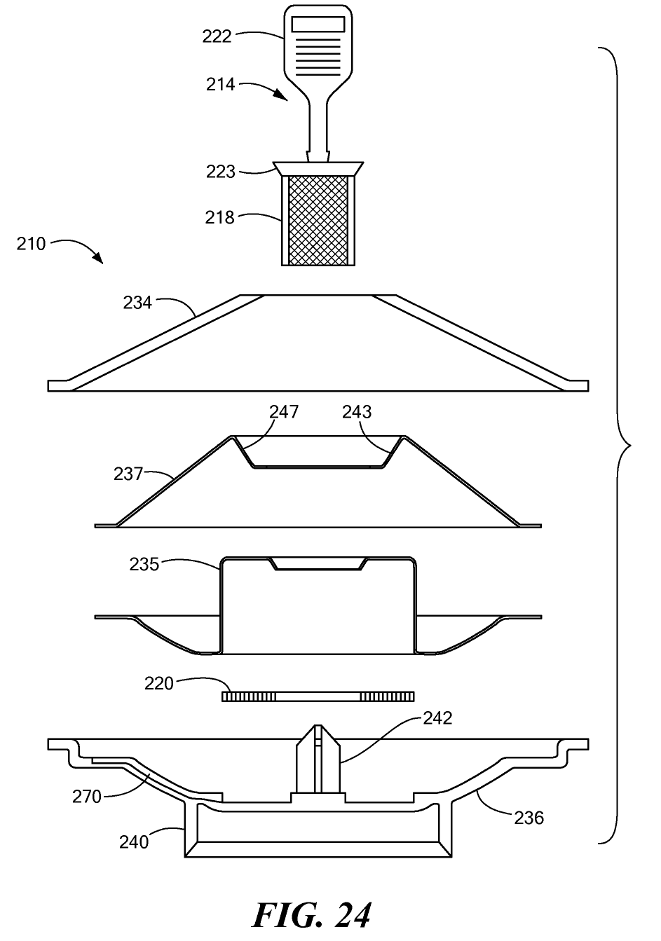

A still further embodiment of a rotor assembly for ova detection is illustrated in FIGS. 23 and 24. In this embodiment, the rotor assembly 210 is generally annular and circular in plan view and coaxial about a central axis 212. The rotor assembly is formed with a housing having a top element 234, a bottom element 236, a mixing chamber element or insert 235, and a sediment or separation chamber element or insert 237. The top and bottom elements are joined and sealed about an annular periphery 238. A central opening 224 is formed through the top element, the sediment chamber insert, and the mixing chamber insert.

The mixing chamber insert 235 is fixed to the bottom surface of the bottom element 236, and includes an upstanding cylindrical wall 229 forming a mixing chamber 226 between the mixing chamber insert and the bottom element. A mixing post or bayonet 242 extends upwardly from a surface of the bottom part into the mixing chamber. An annular rib 240 forming a rotor holder for mounting in a centrifuge depends from the underside of the bottom element.

The sediment chamber insert 237 is also fixed to the bottom surface of the bottom element and forms a sediment chamber 228 between the sediment chamber insert and the upper element. A channel or passage 270 is formed in the bottom surface of the bottom element to provide a passage from the mixing chamber 226 to the sediment chamber 228. A prefiltration screen 220 is disposed in the bottom of the mixing chamber 226 about the mixing post 242 to prevent passage of larger clumps through the channel 270 to the sediment chamber 228. An annular ova collection area or trough 243 is formed by an annular sloped surface 247 of the sediment chamber insert 237 coaxial about the central opening 224.

In operation, a coring assembly 214 with a sample of fecal material is inserted through the opening into the mixing chamber 226 over the mixing post 242. The sample is mixed in a suitable amount of flotation solution. In the embodiment illustrated, the coring assembly includes a coring element 218 having a cylindrical grid surface through which the fecal material passes to mix with the flotation solution. After the sample is fully mixed, the coring assembly is pushed downwardly until an annular flange 223 at the top of the coring element snaps under and seals the opening of the mixing chamber. The handle 222 is then removed, as discussed above.

The rotor assembly 210 is placed in a centrifuge and spun. The fecal matter and flotation solution mixture is forced outwardly to the wall of the mixing chamber 226 and then downwardly through the prefiltration screen 220, which retains larger clumps. The filtered solution enters the small channel 270 adjacent the prefiltration screen 220 and is forced to the base of the sediment chamber 228. The sediment chamber fills with the fecal matter and the flotation solution mixture. After a short high speed spin, ova migrate to the surface of the fluid in the collection trough 243. A small hole may be included in the sediment chamber insert to prevent liquid from escaping the rotor. Excess liquid passes through the small hole under centrifugal force and is contained within the void 245 between the two inserts. At the completion of the centrifugation cycle, ova fall into the collection trough 243.

Flotation solution is added through the central opening 224, if necessary, and a coverslip is placed over the opening. Ova float to the underside of the coverslip after a short time. The coverslip is removed for detection of ova thereon, as discussed above.

A still further embodiment of a rotor assembly for ova detection is illustrated in FIGS. 25. The rotor assembly 310 is generally annular and circular in plan view and coaxial about a central axis 312. The rotor assembly is formed with a housing in three parts: a top element 334, a bottom element 336, and a middle element or insert 335. The top element and the bottom element are joined and sealed at an annular periphery 338. A central opening 324 is formed through the top element. An annular rib 340 forming a rotor holder for mounting in a centrifuge depends from the underside of the bottom element.

The middle insert 335 is fixed to the bottom element 336 and forms a cone-shaped sediment or separation chamber 328 between the insert and the top element 334. One or more fins or other structures (not shown) may be included in the sediment chamber to reduce unwanted remixing of the sample upon deceleration of the rotor assembly.

A circular mixing or receiving chamber 326, which also serves as a collection trough, is formed in the center of the middle insert 335. One or more notches 327 may be provided in the rim of the collection trough to facilitate flow of liquid and displacement air when filling the rotor assembly with a fecal sample and flotation fluid.

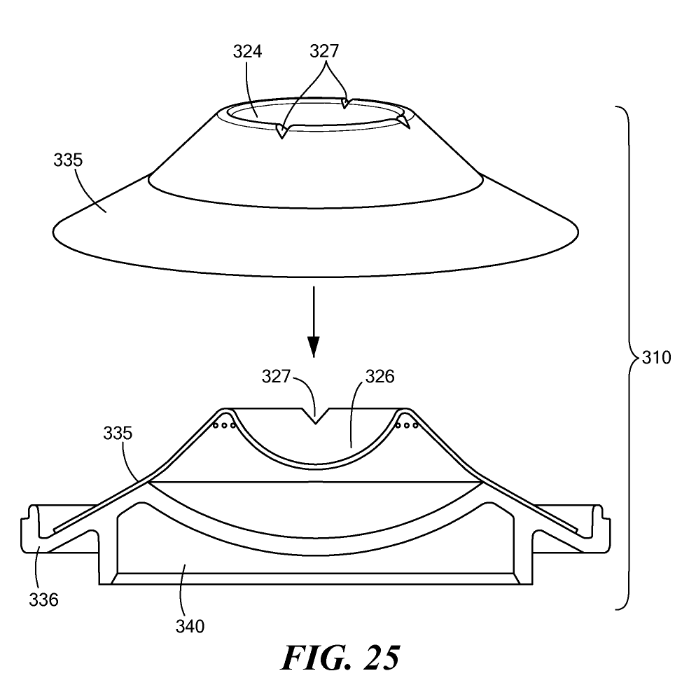

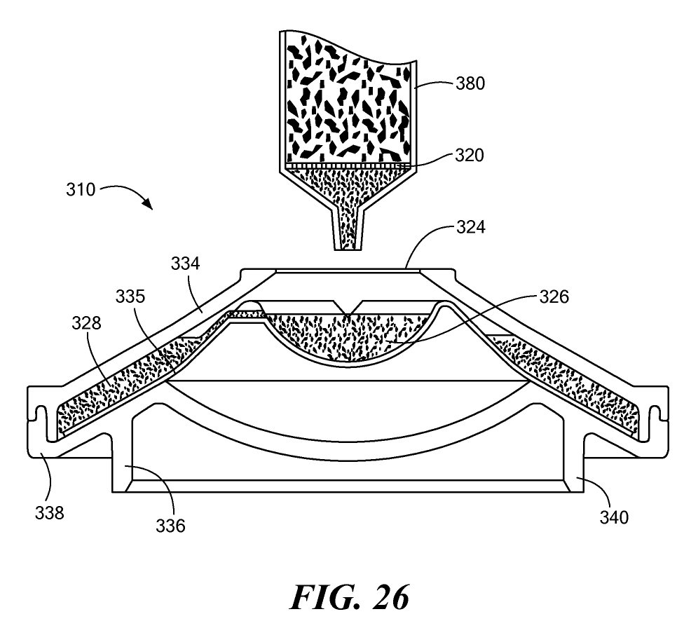

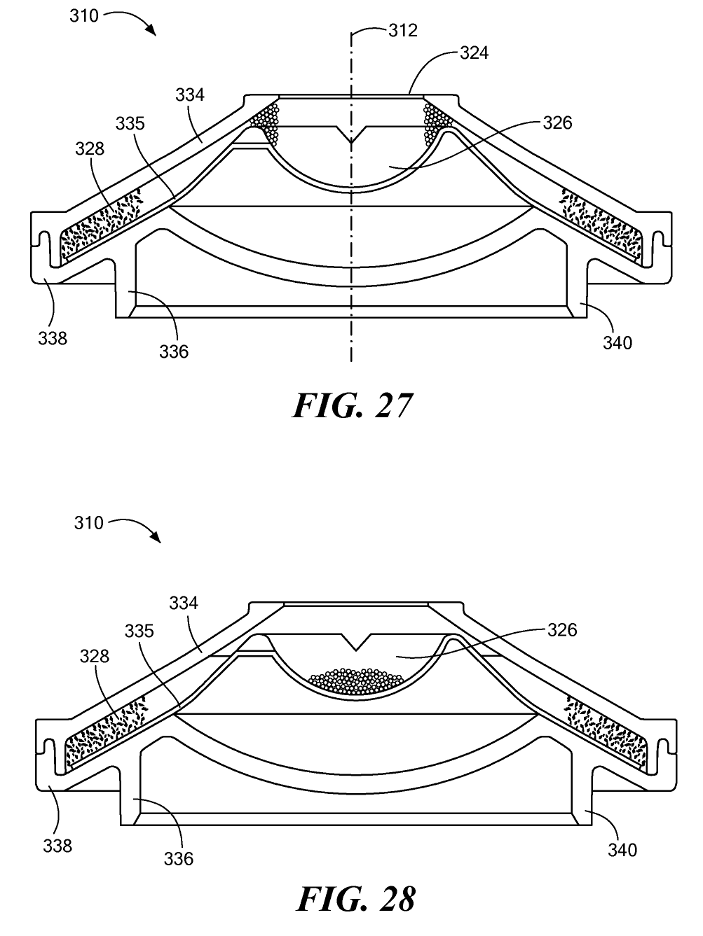

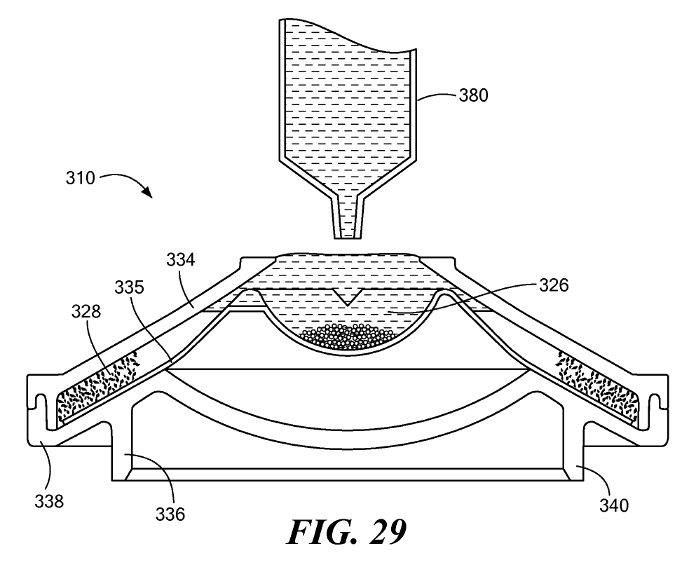

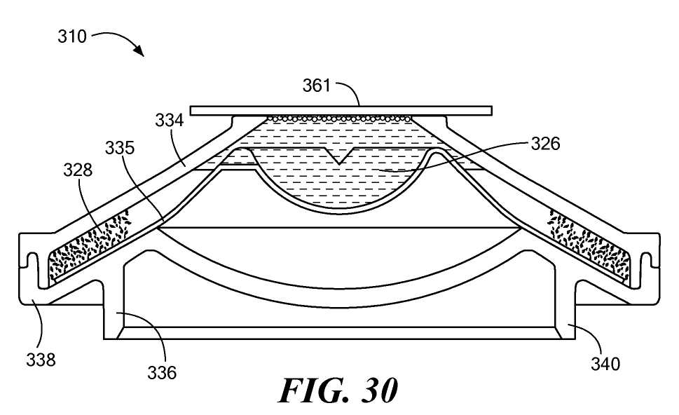

A measured volume of a mixture of a fecal sample and flotation solution is added to the mixing or receiving chamber via a syringe or other measuring device 380 (FIG. 26). A fill line may also be provided on the rotor assembly 310 to ensure the proper volume is added. Preferably, the syringe includes a screen filter 320 to filter out larger clumps. Alternatively, the mixture can be poured through a strainer. The rotor assembly is placed in a centrifuge and spun at a high speed. The fecal matter is forced outwardly in the sediment chamber and the ova are forced inwardly to the surface of the flotation solution (FIG. 27). When the rotor assembly decelerates and stops, the ova are deposited in the collection trough (FIG. 28). Flotation solution is added as necessary through the central opening 324 until a meniscus is formed in the central opening (FIG. 29). A coverslip 361 is placed over the opening. After a short time period, the ova float up to the coverslip, which is then removed for detection of the ova (FIG. 30).

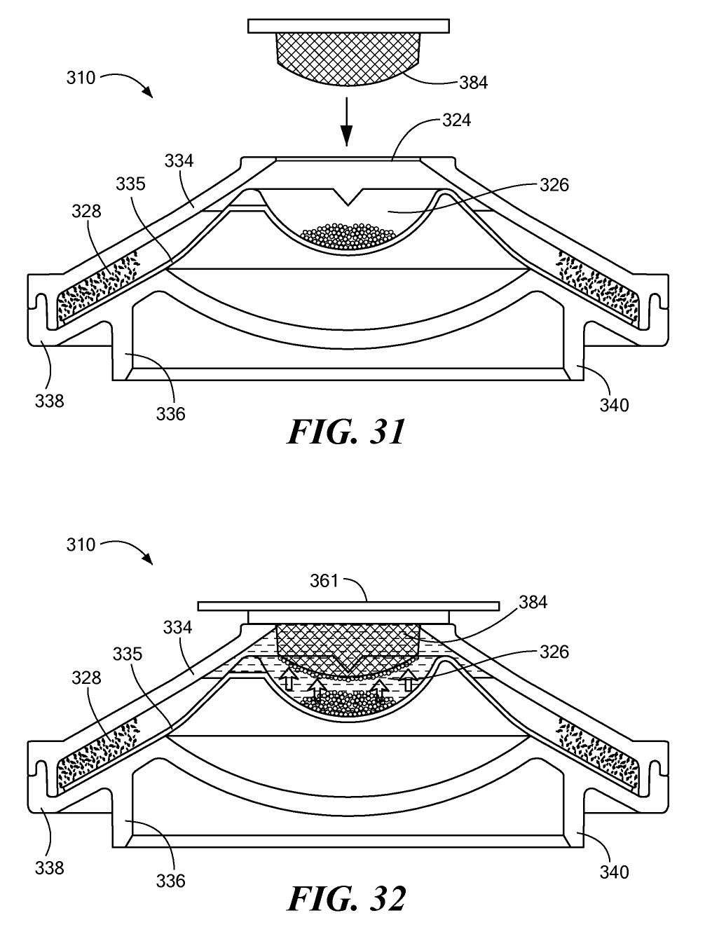

Optionally, a screen 384, illustrated in FIGS. 31 and 32, may be placed in the central opening 324 at the completion of the spin cycle and before the flotation fluid is added. Then the flotation fluid is added. The screen inhibits passage of larger buoyant material to the coverslip 361, while allowing passage of ova therethrough to the coverslip.

The rotor assembly and coring assembly can be formed from any suitable material and by any suitable method. A plastic material is typically used. The rotor assembly may be readily manufactured by vacuum forming or injection molding the various pieces, which can then be assembled by sonic welding or with an adhesive. The rotor assembly is particularly useful for the separation of parasite ova in fecal matter, although it can be used in other applications to harvest buoyant material.

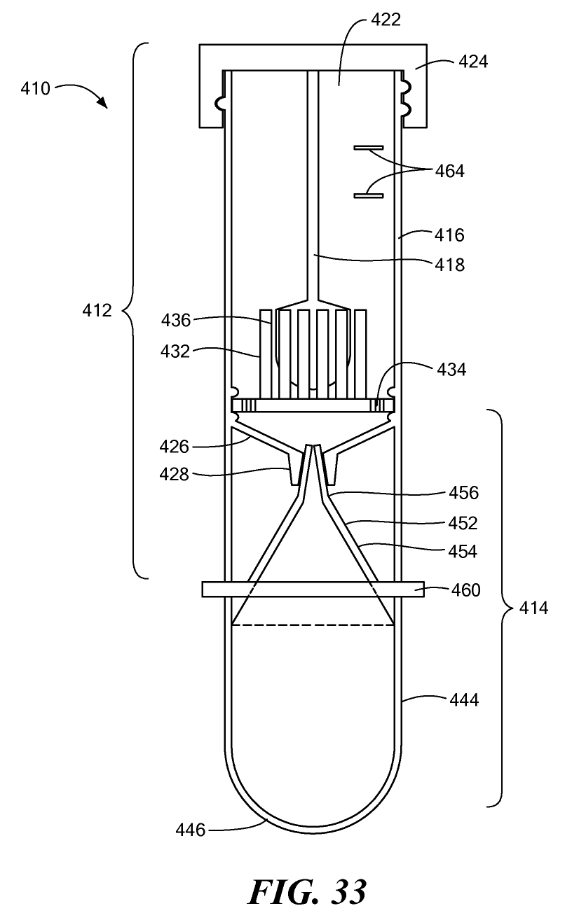

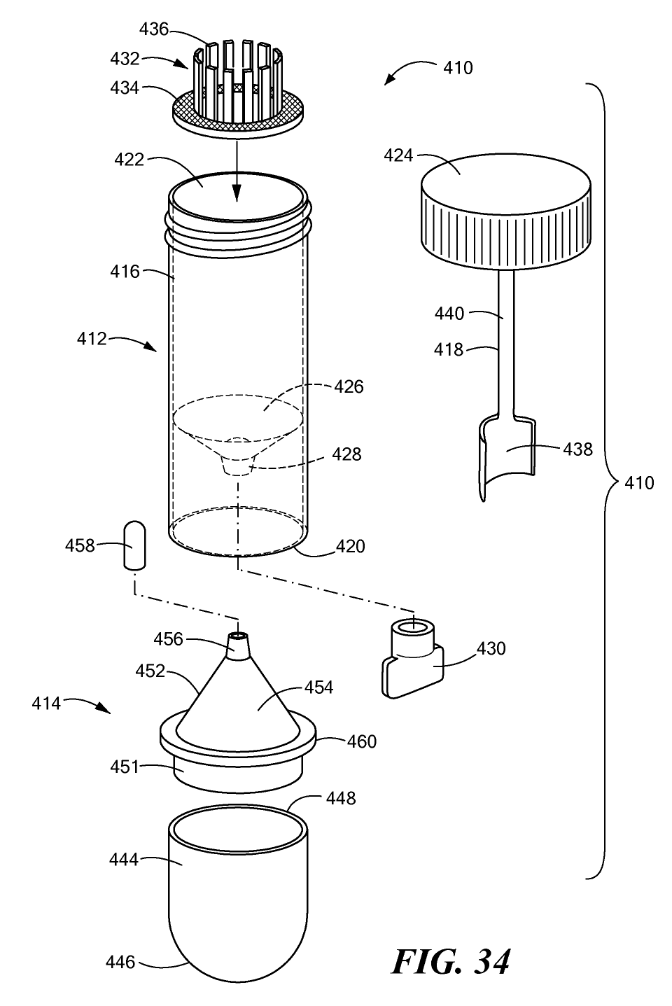

In another aspect of the invention, a centrifugal device is provided in which the ova are delivered to an outlet of a pipette tip for dispensing onto a microscope slide or coverslip. Referring to FIGS. 33-47, the centrifugal device 410 includes a sample collection assembly 412, in which a fecal sample is placed and mixed, and a centrifuge assembly 414, to which the fecal sample is transferred for centrifugation and subsequent dispensing.

The sample collection assembly 412 includes a sample container 416 and a sample collector 418. The sample container is generally tube shaped or cylindrical. One end 420 is open and matable with the centrifuge assembly 414 for transfer of the fecal sample. The opposite end 422 is closable with a cap 424, for example, a screw cap that mates with threads formed at the opposite end 422. A filter funnel 426 is disposed at an internal position within the container. The filter funnel is conical, tapering toward a central axis of the container and includes a central outlet 428. A plug 430 is provided for closing the central outlet 428 during the initial mixing of the fecal sample.

A filter device 432 is seated within the sample container 416 above the filter funnel 426. The filter device 432 includes an annular grid 434 and a disrupter 436 formed of a plurality of upstanding ribs or fingers cylindrically arranged within the grid. The ribs and the grid break up or mash a fecal sample that is placed in the container 416.

The sample collector 418 includes a scoop 438 on a handle 440, which is preferably integrally formed with the cap 424 that closes the end 422 of the sample container 416. A screw cap, snap fit cap or other closure mechanism can be provided, as will be appreciated by those of skill in the art.

The centrifuge assembly 414 includes a flexible tube 444 closed at one end 446 and open at the opposite end 448. A pipette tip 452, which is preferably more rigid than the flexible tube, is attached to the flexible tube at the open end, in any suitable manner, as with a sleeve 451 that fits over the flexible tube. Alternatively, the tube can be a blow-molded part with the pipette tip integral to the tube. The pipette tip includes a funnel shaped portion 454 tapering to a narrow outlet 456. A plug 458 is provided for closing the outlet. A rigid annular collar 460 may be provided for gripping the centrifuge assembly.

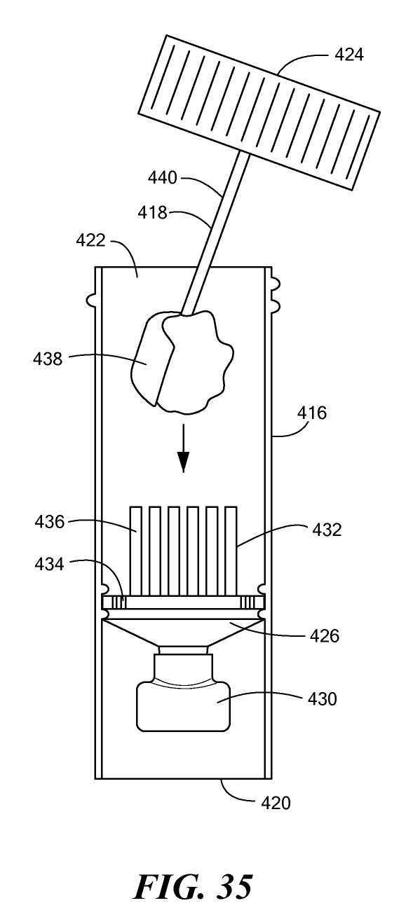

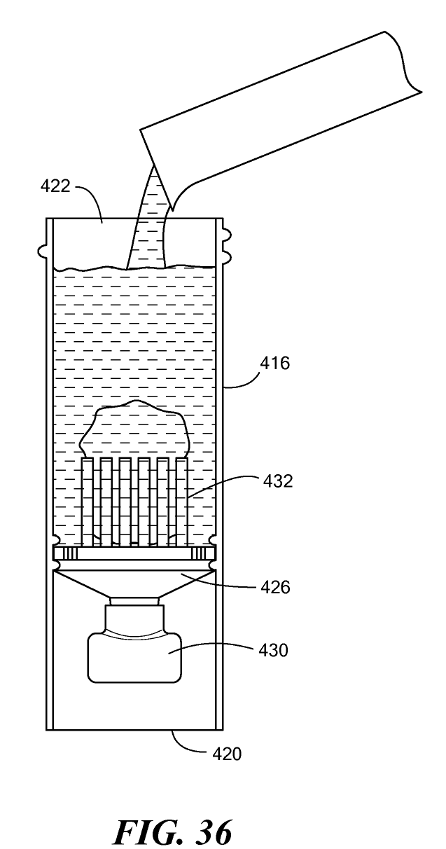

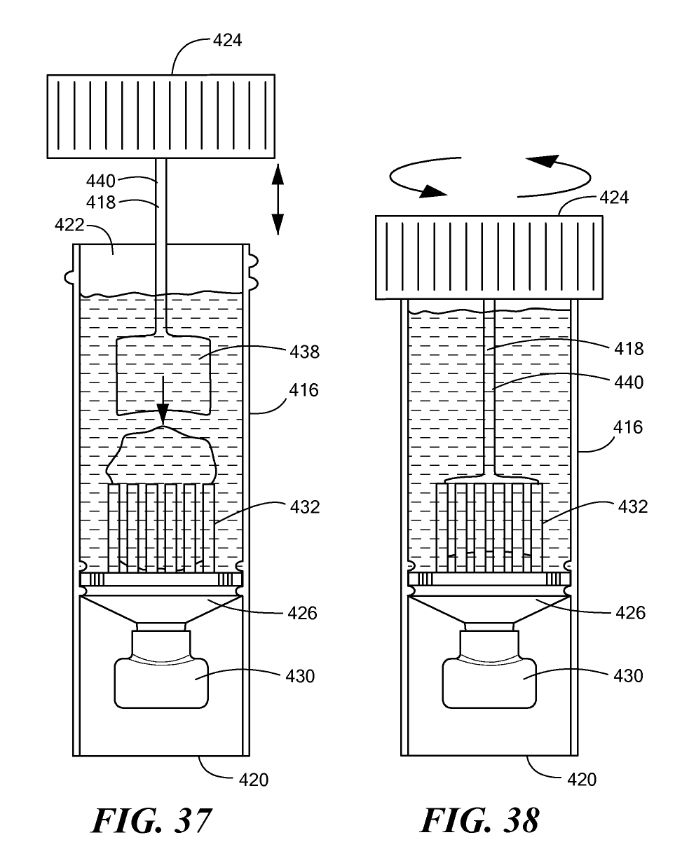

In operation, a fecal sample is collected with the scoop 438 of the sample collector 418 and placed in the sample container 416 (FIG. 35). An appropriate amount of flotation fluid is added to the container (FIG. 36). Maximum and minimum fill lines 464 can be provided on the side of the container 416 to aid the user in adding the flotation fluid (FIG. 33). The user reciprocates and rotates the scoop 438 to break up clumps and release the ova into the fluid (FIG. 37). The container is then sealed by screwing or otherwise affixing the cap 424 to the end (FIG. 38). The container may be shaken to further disrupt the fecal sample.

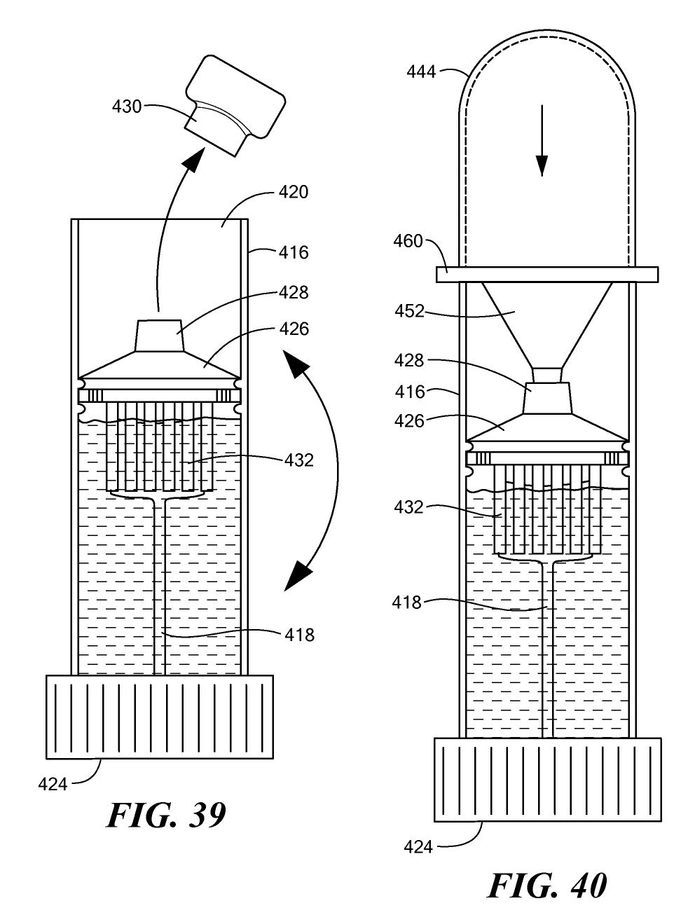

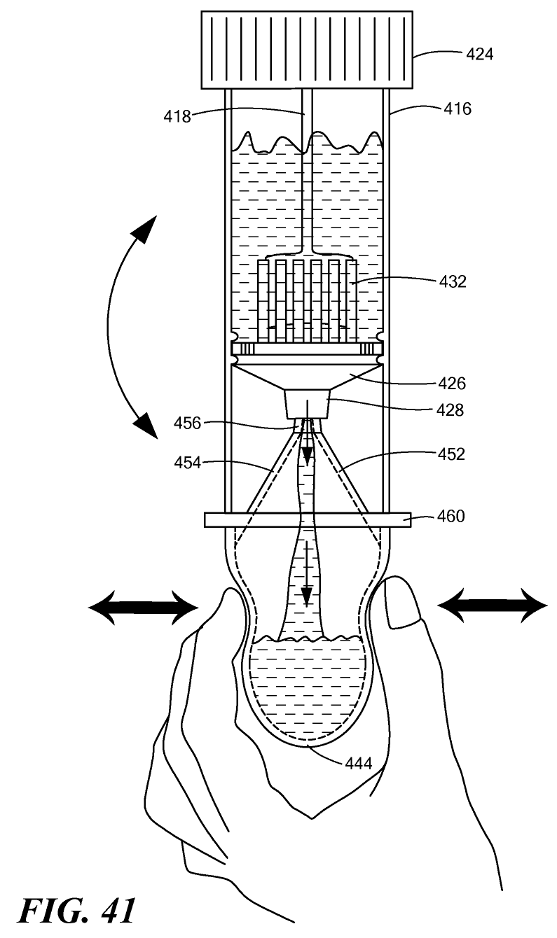

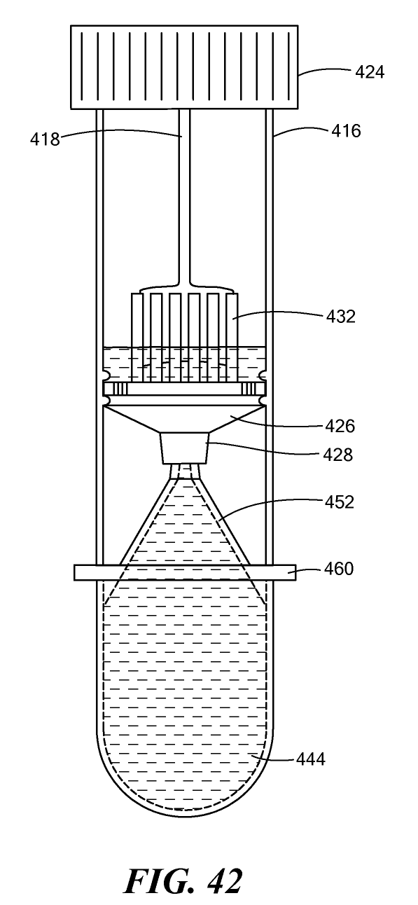

The container 416 is then inverted and placed on a level surface. The plug 430 closing the filter funnel outlet 428 is removed (FIG. 39). The outlet 456 of the pipette top 452 of the centrifuge assembly 414 is then placed into or over the outlet 428 of the filter funnel 426 (FIG. 40). The assembly is then inverted again and the bottom of the flexible tube 444 is gently compressed and released several times to force the sample through the filter grid 434 and the filter funnel outlet 428 into the flexible tube (FIG. 41). Displacement air bubble action may help to further disrupt the sample and prevent clogging of the grid. This action is continued until the tube is filled to the top of the pipette outlet 456 (FIG. 42).

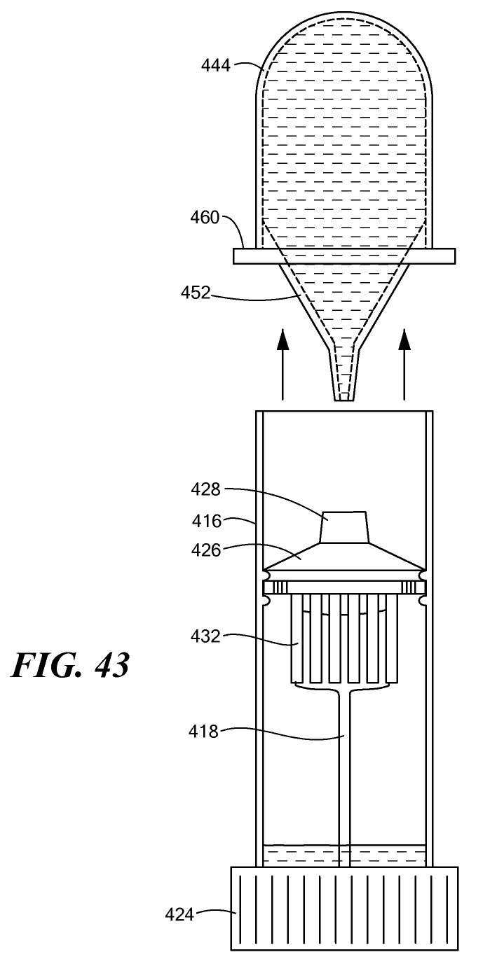

The assembly is inverted once more and the filled flexible tube 444 is removed from the container 416, for example, by grasping the rigid collar 460 (FIG. 43). The container 416 may be recapped and discarded.

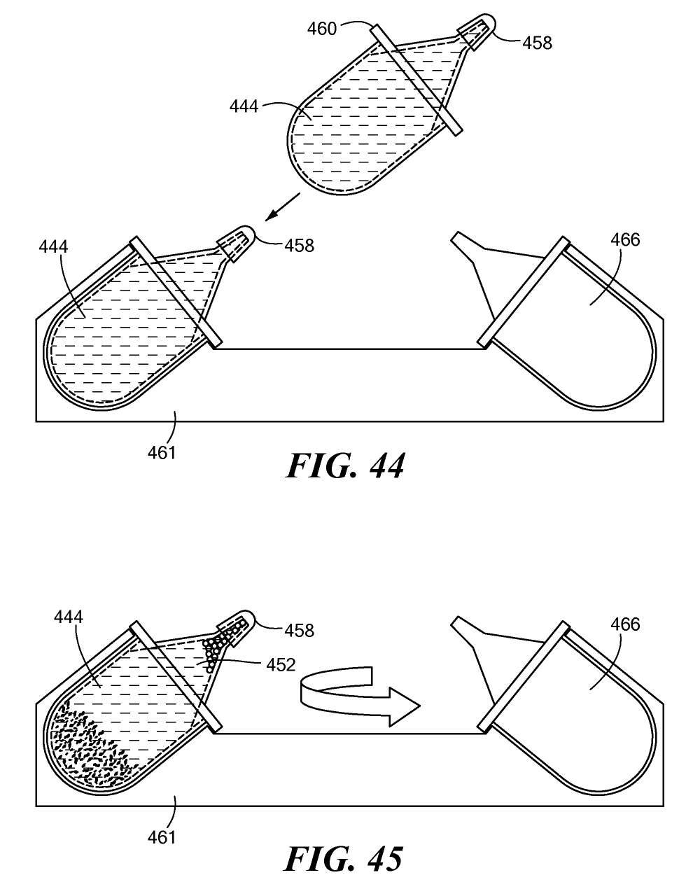

The filled tube 444 is capped with the plug 458 and placed into a centrifuge 461 (FIG. 44). If many samples are prepared in this manner, the centrifuge can be filled with multiple tubes for batch processing of multiple samples. If necessary, a balance tube 466 is added to the centrifuge. The tube is spun at high speeds for a suitable time, such as 30 seconds. Debris is forced to the bottom of the tube. Ova migrate toward the center of rotation and concentrate at the pipette outlet 456 (FIG. 45).

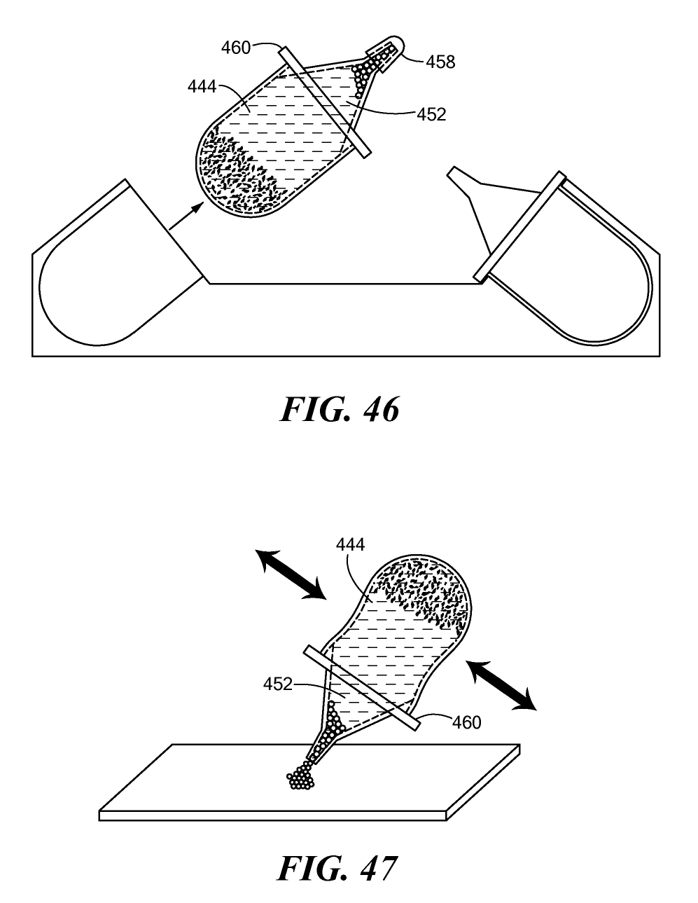

After centrifugation, the tube 444 is carefully removed from the centrifuge, for example, by lifting at the rigid collar 460 (FIG. 46). The plug 458 is removed while holding the tube upright. The tube is carefully inverted and a drop or two of concentrated ova are dispensed onto a microscope slide 468 by gently depressing the sides of the tube (FIG. 47). A coverslip is applied, and the slide is examined using standard microscopy techniques.

Alternatively, after centrifugation, the tube 444 is held upright and a coverslip is placed over the outlet 456. The tube is gently squeezed to dispense the ova at the outlet onto the coverslip, to which the ova adhere. The coverslip is then placed on a slide, and the slide is examined. This procedure is particularly useful if the tube is not completely filled and there is an air bubble present in the tube, as an air bubble could cause the ova to disperse into the fluid when the tube is inverted and before dispensing onto the slide.

The device 410 is a closed system, minimizing exposure by the user to the fecal sample. The procedure using this device can be done rapidly, in just a few minutes. The user does not have to wait for gravity floatation of the ova. The device requires minimal time to set up, and accurate results can be achieved.

The invention is not to be limited by what has been particularly shown and described, except as indicated by the appended claims.

What is claimed is:

1. A centrifugal device for separation of buoyant material comprising:
a rotor assembly having a central axis comprising:
a housing coaxial about the central axis and comprising a top element and a bottom element, an opening centrally located in the top element;
a mixing chamber centrally located within the housing and coaxial about the central axis;
an annular sediment chamber disposed within the housing radially outwardly of the mixing chamber; and
a passage between the mixing chamber and the sediment chamber.

2. The centrifugal device of claim 1, further comprising a coring assembly comprising a lower portion comprising a coring element and a removable upper portion comprising a handle.

3. The centrifugal device of claim 2, wherein the coring element comprises a number of fingers arranged in a circle.

4. The centrifugal device of claim 3, wherein the fingers have tapered ends.

5. The centrifugal device of claim 3, wherein the fingers are wedge-shaped in cross-section.

6. The centrifugal device of claim 2, wherein the lower portion of the coring assembly comprises a filter element.

7. The centrifugal device of claim 2, wherein the filter element is located above the coring element and sized to mate within the opening in the top element.

8. The centrifugal device of claim 2, wherein the filter element is located above the coring element and sized to mate within an opening of the mixing chamber.

9. The centrifugal device of claim 2, wherein the coring element comprises a filter element.

10. The centrifugal device of claim 2, wherein the handle is attached to the coring element by a frictional fit.

11. The centrifugal device of claim 1, wherein the handle is attached to the coring element by a snap fit.

12. The centrifugal device of claim 2, wherein the handle is attached to the coring element by breakaway tabs.

13. The centrifugal device of claim 2, wherein the coring assembly further comprises a locking mechanism to retain the coring element in the rotor assembly.

14. The centrifugal device of claim 13, wherein the locking mechanism comprises an upwardly facing shoulder configured to mate with a corresponding downwardly facing surface in the housing.

15. The centrifugal device of claim 2, wherein the coring assembly further comprises a flapper valve sized to fit over an opening of the mixing chamber.

16. The centrifugal device of claim 1, further comprising at least one fill port extending from the top element of the housing to a location near a bottom inner surface of the mixing chamber in the housing.

17. The centrifugal device of claim 1, further comprising a mixing post extending upwardly into the mixing chamber from a bottom surface of the housing and coaxial about the central axis.

18. The centrifugal device of claim 1, further comprising one or more radial vanes within the mixing chamber.

19. The centrifugal device of claim 1, further comprising one or more radial vanes within the sediment chamber.

20. The centrifugal device of claim 1, further comprising a collection trough disposed within the housing radially inwardly of the sediment chamber.

21. The centrifugal device of claim 20, wherein the collection trough coaxially surrounds the mixing chamber.

22. The centrifugal device of claim 1, wherein the passage between the mixing chamber and the sediment chamber comprises a capillary gap.

23. The centrifugal device of claim 1, further comprising a prefiltration screen in the mixing chamber before the passage to the sediment chamber.

24. The centrifugal device of claim 1, further comprising a middle element disposed between the top element and the bottom element, an overflow chamber formed between the top element and the middle element.

25. The centrifugal device of claim 1, wherein the sediment chamber is formed between the middle element and the bottom element.

26. The centrifugal device of claim 1, further comprising a mixing chamber insert and a sediment chamber insert disposed between the top element and the bottom element, the mixing chamber formed between the mixing chamber insert and the bottom element, the sediment chamber formed between the sediment chamber insert and the top element.

27. The centrifugal device of claim 1, further comprising an overspillage lip formed on an upper surface of the housing.

28. The centrifugal device of claim 1, further comprising a cap removably disposed over the central opening.

29. The centrifugal device of claim 1, further comprising a cap removably disposed over the rotor holder element.

30. The centrifugal device of claim 1, further comprising a coverslip sized to rest on the central opening.

31. The centrifugal device of claim 1, further comprising a rotor holder element configured for mounting the rotor assembly in a centrifuge for rotation about the central axis.

32. The centrifugal device of claim 31, wherein the rotor holder element comprises an annular rib extending from the bottom element of the housing to affix the rotor assembly to the centrifuge.

33. A method for separating buoyant material comprising:
providing a centrifugal device comprising a rotor assembly having a central axis and a housing coaxial and rotatable about the central axis, a central opening in the housing, a mixing chamber centrally located in the housing and coaxial about the central axis and an annular sediment chamber radially outwardly of the mixing chamber;
mixing a sample of matter to be separated with a flotation fluid in the mixing chamber of the rotor assembly;
spinning the rotor assembly in a centrifuge about the central axis, forcing matter more dense than the flotation fluid radially outwardly into the sediment chamber and matter less dense than the flotation fluid inwardly to the surface of the fluid;
stopping the spinning of the rotor assembly;
placing a coverslip over the central opening; and
allowing the less dense matter to adhere to the coverslip.

34. The method of claim 33, further comprising filling the rotor assembly with additional flotation fluid after the spinning step to form a meniscus at the central opening in the housing.

35. The method of claim 34, further comprising filling the rotor assembly with fluid to form a meniscus from the bottom of the rotor assembly to avoid disrupting a fluid surface.

36. The method of claim 33, further comprising filling the rotor assembly with sufficient fluid to form a meniscus at the central opening in the housing prior to spinning the rotor assembly.

37. The method of claim 33, further comprising coring the sample of matter with a coring assembly and inserting the coring assembly through the central opening in the rotor assembly.

38. The method of claim 37, further comprising mixing the sample of matter by rotating or reciprocating or rotating and reciprocating the coring assembly in the mixing chamber;

39. The method of claim 37, further comprising removing a handle from the coring assembly after mixing the sample of matter.

40. The method of claim 33, wherein the sample of matter to be separated comprises a fecal sample.

41. The method of claim 33, wherein the flotation fluid comprises a fluid having a specific gravity less than a specific gravity of a fecal sample and greater than a specific gravity of parasite ova.

42. A centrifugal device for separation of buoyant material comprising:
a sample collection assembly comprising:
a container,
a chamber located within the container, and
an outlet from the chamber; and
a centrifuge assembly comprising:
a tube closed at one end, the tube receivable in a centrifuge, and
a pipette top disposed at an opposite end of the tube, the pipette top matable with the outlet from the chamber for receiving material from the chamber.

43. The centrifugal device of claim 42, wherein the sample collection assembly further comprises a sample collector for collecting a sample of material to be separated and for depositing the sample of material in the container.

44. The centrifugal device of claim 43, wherein the sample collector comprises a scoop attached to one end of a handle, and a cap attached to an opposite end of the handle, the cap configured to close an end of the container.

45. The centrifugal device of claim 42, further comprising a funnel element disposed within the container, the outlet comprising a narrowed outlet of the funnel element.

46. The centrifugal device of claim 42, further comprising a filter device disposed within the container upstream of the outlet.

47. The centrifugal device of claim 46, wherein the filter device comprises a grid.

48. The centrifugal device of claim 46, wherein the filter device comprises a plurality of cylindrically arranged upstanding ribs.

49. The centrifugal device of claim 42, wherein the tube is flexible to allow compression thereof.

50. A method for separating buoyant material comprising:
providing a centrifugal device comprising a container and a centrifuge assembly matable with the container, the centrifuge assembly comprising a tube and a pipette tip;
mixing a sample of matter to be separated with a flotation fluid in the container;
transferring the sample of matter to the centrifuge assembly while mated to the container;
spinning the centrifuge assembly in a centrifuge, forcing matter more dense than the flotation fluid downwardly in the tube and matter less dense than the flotation fluid upwardly toward the pipette tip;
stopping the spinning of the centrifuge assembly; and
dispensing the less dense matter from the pipette tip.

51. The method of claim 50, further comprising dispensing the less dense matter from the pipette tip by inverting the centrifuge assembly and dispensing a few drops onto a microscope slide.

52. The method of claim 50, further comprising dispensing the less dense matter from the pipette tip by placing a coverslip over the top and allowing the less dense matter to adhere to the coverslip.

53. The method of claim 50, further comprising filtering the sample of matter in the container prior to transferring the sample to the centrifuge assembly.

54. The method of claim 50, wherein the sample of matter to be separated comprises a fecal sample.

55. The method of claim 50, wherein the flotation fluid comprises a fluid having a specific gravity less than a specific gravity of a fecal sample and greater than a specific gravity of parasite ova.

56. The centrifugal device of claim 1, further comprising a plurality of dispersion elements in the sediment chamber.
